SEQUENCE LISTING (22 sequences)

SEQ ID NO: 1 - DNA, 25 bp, Artificial sequence
  Single strand DNA oligonucleotide
agtggcaatgcttagactctttcgt

SEQ ID NO: 2 - DNA, 25 bp, Artificial sequence
  Single strand DNA oligonucleotide
gcgtgccagacagagtgctaggcat

SEQ ID NO: 3 - DNA, 36 bp, Artificial sequence
  Single strand DNA oligonucleotide
gaggatccaagctttgttacaaaggggcgaccgcgt

SEQ ID NO: 4 - DNA, 2214 bp, Homo sapiens
gcgtgccagacagagtgctaggcatgggggcagaggtgaatcagatgacagccacctctcaccacgaggagtggctgaaagtgtgactggactacaggcaatcctggccttggcagggagtggggccagccagcagaaacagtgggctgtacaacatcaccttcaaatatgacaattgtaccacctacttgaatccagtggggaagcatgtgattgctgacgcccagaatatcaccatcagccagtatgcttgccatgaccaagtggcagtcaccattctttggtccccaggggccctcggcatcgaattcctgaaaggatttcgggtaatactggaggagctgaagtcggagggaagacagtgccaacaactgattctaaaggatccgaagcagctcaacagtagcttcaaaagaactggaatggaatctcaacctttcctgaatatgaaatttgaaacggattatttcgtaaaggttgtcccttttccttccattaaaaacgaaagcaattaccaccctttcttctttagaacccgagcctgtgacctgttgttacagccggacaatctagcttgtaaacccttctggaagcctcggaacctgaacatcagccagcatggctcggacatgcaggtgtccttcgaccacgcaccgcacaacttcggcttccgtttcttctatcttcactacaagctcaagcacgaaggacctttcaagcgaaagacctgtaagcaggagcaaactacagagatgaccagctgcctccttcaaaatgtttctccaggggattatataattgagctggtggatgacactaacacaacaagaaaagtgatgcattatgccttaaagccagtgcactccccgtgggccgggcccatcagagccgtggccatcacagtgccactggtagtcatatcggcattcgcgacgctcttcactgtgatgtgccgcaagaagcaacaagaaaatatatattcacatttagatgaagagagctctgagtcttccacatacactgcagcactcccaagagagaggctccggccgcggccgaaggtctttctctgctattccagtaaagatggccagaatcacatgaatgtcgtccagtgtttcgcctacttcctccaggacttctgtggctgtgaggtggctctggacctgtgggaagacttcagcctctgtagagaagggcagagagaatgggtcatccagaagatccacgagtcccagttcatcattgtggtttgttccaaaggcatgaagtactttgtggacaagaagaactacaaacacaaaggaggtggccgaggctcggggaaaggagagctcttcctggtggcggtgtcagccattgccgaaaagctccgccaggccaagcagagttcgtccgcggcgctcagcaagtttatcgccgtctactttgattattcctgcgagggagacgtccccggtatcctagacctgagtaccaagtacagactcatggacaatcttcctcagctctgttcccacctgcactcccgagaccacggcctccaggagccggggcagcacacgcgacagggcagcagaaggaactacttccggagcaagtcaggccggtccctatacgtcgccatttgcaacatgcaccagtttattgacgaggagcccgactggttcgaaaagcagttcgttcccttccatcctcctccactgcgctaccgggagccagtcttggagaaatttgattcgggcttggttttaaatgatgtcatgtgcaaaccagggcctgagagtgacttctgcctaaaggtagaggcggctgttcttggggcaaccggaccagccgactcccagcacgagagtcagcatgggggcctggaccaagacggggaggcccggcctgcccttgacggtagcgccgccctgcaacccctgctgcacacggtgaaagccggcagcccctcggacatgccgcgggactcaggcatctatgactcgtctgtgccctcatccgagctgtctctgccactgatggaaggactctcgacggaccagacagaaacgtcttccctgacggagagcgtgtcctcctcttcaggcctgggtgaggaggaacctcctgcccttccttccaagctcctctcttctgggtcatgcaaagcagatcttggttgccgcagctacactgatgaactccacgcggtcgcccctttgtaacaaa

SEQ ID NO: 5 - protein, 739 aa, Homo sapiens [sequence omitted]

SEQ ID NO: 6 - protein, 707 aa, Homo sapiens
LDYRQSWPWQGVGPASRNSGLYNITFKYDNCTTYLNPVGKHVIADAQNITIRQYACHDQVAVTILWSPGALGIEFLKGFRVILEELKSEGRQCQQLILKDPKQLNSSFKRTGMESQPFLNMKFETDYFVKVVPFPSIKNESNYHPFFFRTRACDLLLQPDNLACKPFWKPRNLNISQHGSDMQVSFDHAPHNFGFRFFYLHYKLKHEGPFKRKTCKQEQTTEMTSCLLQNVSPGDYIIELVDDTNTTRKVMHYALKPVHSPWAGPIRAVAITVPLVVISAFATLFTVMCRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 7 - DNA, 2339 bp, Homo sapiens
agcggattcgctcttcttttcctccgggaaaagaaacgggaagtggccgtgggccggtgaattccgtgtagtggccaagcttgttccaaagagggggaggtgttgacagtctcttgcccactgaagcgtgccagacagagtgctaggcatgggggcagaggtgaatcagatgacagccacctctcaccacgaggagtggctgaaagtgtgactggactacaggcaatcctggccttggcagggagtggggccagccagcagaaacagtgggctgtacaacatcaccttcaaatatgacaattgtaccacctacttgaatccagtggggaagcatgtgattgctgacgcccagaatatcaccatcaggcagtatgcttgccatgaccaagtggcagtcaccattctttggtccccaggggccctcggcatcgaattcctgaaaggatttcgggtaatactggaggagctgaagtcggagggaagacagtgccaacaactgattctaaaggatccgaagcagctcaacagtagcttcaaaagaactggaatggaatctcaacctttcctgaatatgaaatttgaaacggattatttcgtaaaggttgtcccttttccttccattaaaaacgaaagcaattaccaccctttcttctttagaacccgagcctgtgacctgttgttacagccggacaatctagcttgtaaacccttctggaagcctcggaacctgaacatcagccagcatggctcggacatgcaggtgtccttcgaccacgcaccgcacaacttcggcttccgtttcttctatcttcactacaagctcaagcacgaaggacctttcaagcgaaagacctgtaagcaggagcaaactacagagatgaccagctgcctccttcaaaatgtttctccaggggattatataattgagctggtggatgacactaacacaacaagaaaagtgatgcattatgccttaaagccagtgcactccccgtgggccgggcccatcagagccgtggccatcacagtgccactggtagtcatatcggcattcgcgacgctcttcactgtgatgtgccgcaagaagcaacaagaaaatatatattcacatttagatgaagagagctctgagtcttccacatacactgcagcactcccaagagagaggctccggccgcggccgaaggtctttctctgctattccagtaaagatggccagaatcacatgaatgtcgtccagtgtttcgcctacttcctccaggacttctgtggctgtgaggtggctctggacctgtgggaagacttcagcctctgtagagaagggcagagagaatgggtcatccagaagatccacgagtcccagttcatcattgtggtttgttccaaaggtatgaagtactttgtggacaagaagaactacaaacacaaaggaggtggccgaggctcggggaaaggagagctcttcctggtggcggtgtcagccattgccgaaaagctccgccaggccaagcagagttcgtccgcggcgctcagcaagtttatcgccgtctactttgattattcctgcgagggagacgtccccggtatcctagacctgagtaccaagtacagactcatggacaatcttcctcagctctgttcccacctgcactcccgagaccacggcctccaggagccggggcagcacacgcgacagggcagcagaaggaactacttccggagcaagtcaggccggtccctatacgtcgccatttgcaacatgcaccagtttattgacgaggagcccgactggttcgaaaagcagttcgttcccttccatcctcctccactgcgctaccgggagccagtcttggagaaatttgattcgggcttggttttaaatgatgtcatgtgcaaaccagggcctgagagtgacttctgcctaaaggtagaggcggctgttcttggggcaaccggaccagccgactcccagcacgagagtcagcatgggggcctggaccaagacggggaggcccggcctgcccttgacggtagcgccgccctgcaacccctgctgcacacggtgaaagccggcagcccctcggacatgccgcgggactcaggcatctatgactcgtctgtgccctcatccgagctgtctctgccactgatggaaggactctcgacggaccagacagaaacgtcttccctgacggagagcgtgtcctcctcttcaggcctgggtgaggaggaacctcctgcccttccttccaagctcctctcttctgggtcatgcaaagcagatcttggttgccgcagctacactgatgaactccacgcggtcgcccctttgtaacaaa

SEQ ID NO: 8 - DNA, 234 bp, Homo sapiens
tgaagcgggcagaaagagtggtggatgatgtccggggactggcatgaccctgggtctcagcagtgctgcttgcatttggactccatggggctttgtgttggaagagcaaattggcttcactctgcatcatgttctcttgttttcccacagggagtggggccagccagcagaaacagtgggctgtacaacatcaccttcaaatatgacaattgtaccacctacttgaatccagtg

SEQ ID NO: 9 - DNA, 585 bp, Homo sapiens
gcggccgccgcggccaccgcccactcggggctggccagcggcgggcggccggggcgcagagaacggcctggctgggcgagcgcacggccatggccccgtggctgcagctctgctccgtcttctttacggtcaacgcctgcctcaacggctcgcagctggctgtggccgctggcgggtccggccgcgcgcggggcgccgacacctgtggctggagggtaaggcgagggcggcgggtttcttgccgtcgccaactcgcggggaacgcagcgcgcacaggtgctcgcggggaggcgagcccgcgccaacctgtctgctcttcgcggggtccgcggccggcctgggtctcactcctcccgcgcatcctcctggtttccctccccggacgcgtgtcctccggccctggccgagatgaaagcggctgcccgaccccggctttgtgttgctaatgagggagtggggccagccagcagaaacagtgggctgtacaacatcaccttcaaatatgacaattgtaccacctacttgaatccagtggggaagcatgtgattgctgacgcccagaatatcaccatcagccagtatgct

SEQ ID NO: 10 - DNA, 2240 bp, Homo sapiens
gaggatcctgacggccatggccccgtggctgcagctctgctccgtcttctttacggtcaacgcctgcctcaacggctcgcagctggctgtggccgctggcgggtccggccgcgcgcggggcgccgacacctgtggctggaggggagtggggccagccagcagaaacagtgggctgtacaacatcaccttcaaatatgacaattgtaccacctacttgaatccagtggggaagcatgtgattgctgacgcccagaatatcaccatcagccagtatgcttgccatgaccaagtggcagtcaccattctttggtccccaggggccctcggcatcgaattcctgaaaggatttcgggtaatactggaggagctgaagtcggagggaagacagtgccaacaactgattctaaaggatccgaagcagctcaacagtagcttcaaaagaactggaatggaatctcaacctttcctgaatatgaaatttgaaacggattatttcgtaaaggttgtcccttttccttccattaaaaacgaaagcaattaccaccctttcttctttagaacccgagcctgtgacctgttgttacagccggacaatctagcttgtaaacccttctggaagcctcggaacctgaacatcagccagcatggctcggacatgcaggtgtccttcgaccacgcaccgcacaacttcggcttccgtttcttctatcttcactacaagctcaagcacgaaggacctttcaagcgaaagacctgtaagcaggagcaaactacagagatgaccagctgcctccttcaaaatgtttctccaggggattatataattgagctggtggatgacactaacacaacaagaaaagtgatgcattatgccttaaagccagtgcactccccgtgggccgggcccatcagagccgtggccatcacagtgccactggtagtcatatcggcattcgcgacgctcttcactgtgatgtgccgcaagaagcaacaagaaaatatatattcacatttagatgaagagagctctgagtcttccacatacactgcagcactcccaagagagaggctccggccgcggccgaaggtctttctctgctattccagtaaagatggccagaatcacatgaatgtcgtccagtgtttcgcctacttcctccaggacttctgtggctgtgaggtggctctggacctgtgggaagacttcagcctctgtagagaagggcagagagaatgggtcatccagaagatccacgagtcccagttcatcattgtggtttgttccaaaggcatgaagtactttgtggacaagaagaactacaaacacaaaggaggtggccgaggctcggggaaaggagagctcttcctggtggcggtgtcagccattgccgaaaagctccgccaggccaagcagagttcgtccgcggcgctcagcaagtttatcgccgtctactttgattattcctgcgagggagacgtccccggtatcctagacctgagtaccaagtacagactcatggacaatcttcctcagctctgttcccacctgcactcccgagaccacggcctccaggagccggggcagcacacgcgacagggcagcagaaggaactacttccggagcaagtcaggccggtccctatacgtcgccatttgcaacatgcaccagtttattgacgaggagcccgactggttcgaaaagcagttcgttcccttccatcctcctccactgcgctaccgggagccagtcttggagaaatttgattcgggcttggttttaaatgatgtcatgtgcaaaccagggcctgagagtgacttctgcctaaaggtagaggcggctgttcttggggcaaccggaccagccgactcccagcacgagagtcagcatgggggcctggaccaagacggggaggcccggcctgcccttgacggtagcgccgccctgcaacccctgctgcacacggtgaaagccggcagcccctcggacatgccgcgggactcaggcatctatgactcgtctgtgccctcatccgagctgtctctgccactgatggaaggactctcgacggaccagacagaaacgtcttccctgacggagagcgtgtcctcctcttcaggcctgggtgaggaggaacctcctgcccttccttccaagctcctctcttctgggtcatgcaaagcagatcttggttgccgcagctacactgatgaactccacgcggtcgcccctttgtaacaaa

SEQ ID NO: 11 - DNA, 23 bp, Artificial sequence
  Single strand DNA oligonucleotide
agcaagtcaggccggtccctata

SEQ ID NO: 12 - DNA, 22 bp, Artificial sequence
  Single strand DNA oligonucleotide
ggtgaaggtcggagtcaacgga

SEQ ID NO: 13 - DNA, 22 bp, Artificial sequence
  Single strand DNA oligonucleotide
gagggatctcgctcctggaaga

SEQ ID NO: 14 - protein, 115 aa, Artificial sequence
  Partial predicted amino acid sequence of hSef-d
AVANSRGTQRAQVLAGRRARANLSALRGVRGRPGSHSSRASSWFPSPDACPPALAEMKAAARPRLCVANEGVGPASRNSGLYNITFKYDNCTTYLNPVGKHVIADAQNITISQYA

SEQ ID NO: 15 - protein, 77 aa, Homo sapiens
SGQKEWWMMSGDWHDPGSQQCCLHLDSMGLCVGRANWLHSASCSLVFPQGVGPASRNSGLYNITFKYDNCTTYLNPV

SEQ ID NO: 16 - DNA, 37 bp, Artificial sequence
  Single strand DNA oligonucleotide
gaggatcctgacggccatggccccgtggctgcagctc

SEQ ID NO: 17 - DNA, 24 bp, Artificial sequence
  Single strand DNA oligonucleotide
tgaagctactgttgagctgcttcg

SEQ ID NO: 18 - DNA, 24 bp, Artificial sequence
  Single strand DNA oligonucleotide
cagacgagtcatagatgcctgagt

SEQ ID NO: 19 - DNA, 25 bp, Artificial sequence
  Single strand DNA oligonucleotide
cttcactctgcatcatgttctcttg

SEQ ID NO: 20 - DNA, 23 bp, Artificial sequence
  Single strand DNA oligonucleotide
gtgactgccacttggtcatggca

SEQ ID NO: 21 - DNA, 23 bp, Artificial sequence
  Single strand DNA oligonucleotide
gacacctgtggctggagggtaag

SEQ ID NO: 22 - DNA, 293 bp, Artificial sequence
  hSef-b RACE derived fragment
aaacgggaagtggccgtgggccggtgaattccgtgtagtggccaagctttgttccaaagagggggaggtgttgacagtctcttgcccactgaagcgtgccagacagagtgctaggcatgggggcagaggtgaatcagatgacagccacctctcaccacgaggagtggctgaaagtgtgactggactacaggcaatcctggccttggcagggagtggggccagccagcagaaacagtgggctgtacaacatcaccttcaaatatgacaattgtaccacctacttgaatccagtg

(19) United States
(12) Patent Application Publication
(10) Pub. No.: US 2006/0079444 A1
(43) Pub. Date: Apr. 13, 2006

(54) Human Sef isoforms and methods of using same for cancer gene therapy

(76) Inventor: Dina Ron, Kiryat-Tivon (IL)

Correspondence Address: ANTHONY CASTORINA, SUITE 207, 2001 JEFFERSON DAVIS HIGHWAY, ARLINGTON, VA, 22202, US

(21) Appl. No.: 10/963,439

(22) Filed: Oct. 11, 2004

(51) Int. Cl. A61K 38/17

(52) U.S. Cl. 514/2

(57) ABSTRACT

A method and pharmaceutical compositions useful for inhibiting the growth of solid tumors are provided. Specifically, the method is effected by administering to a subject in need thereof an agent capable of upregulating the expression level and/or activity of at least a functional portion of Sef, wherein the functional portion being capable of inhibiting RTK-mediated cell proliferation.

### FIELD AND BACKGROUND OF THE INVENTION

The present invention relates to polypeptides and polynucleotides expressing the human Sef b-d isoforms (hSefb-d), and more particularly, to the use of such isoforms in the inhibition of uncontrolled malignant proliferation of solid tumors.

Solid tumors account for the majority of human tumors and among them, carcinomas of an epithelial origin, account for over 80%. Conventional therapies for solid tumors involves the administration of anti-tumor drugs such as thymidylate synthase inhibitors (e.g., 5-fluorouracil; Rose M G et al., 2002; Clin Colorectal Cancer. 1: 220-9), nucleoside analogs [e.g., gemcitabine (Gemzar); Seidman A D., 2001. Oncology (Huntingt). 15: 11-14), non-steroidal (e.g., anastrozole and letrozole) and steroidal (exemestane) aromatase inhibitors (Lake D E and Hudis C., 2002; Cancer Control; 9: 490-8), taxanes and topoisomerase-I inhibitors (e.g., irinotecan; Van Cutsem, E. 2004; The Oncologist, 9, Suppl 2, 9-15). However, the use of such drugs often fails due to the development of drug resistance by the cancer cells. Thus, despite the tremendous progress in understanding tumor biology and early detection of cancer, cancer mortality rates have not been significantly reduced.

The growth of solid tumors depends on nutrients and oxygen which are supplied by the tumor vasculature. The growth of new blood vessels into the tumor is controlled by paracrine signals, many of which are mediated by protein ligands which modulate the activity of transmembrane tyrosine kinase receptors (RTK). These include vascular endothelial growth factor (VEGF) and its receptor families (VEGFR-1, VEGFR-2, neuropilin-1 and neuropilin-2), Angiopoietins 1-4 (Ang-1, Ang-2) and their respective receptors (Tie-1 and Tie-2), basic fibroblast growth factor (bFGF), platelet derived growth factor (PDGF), transforming growth factor β (TGF-β) and their receptors. Thus, it was conceivable that inhibition of angiogenesis can inhibit the growth of solid tumors.

Indeed, several studies have demonstrated that anti-angiogenic agents such as Endostatin, AGM-1470, angiostatin, BB-94, 2-Methoxyestradiol (2-ME) and Taxol in can inhibit tumor growth in vivo (Boehm et al., 1997; Nature; 390: 404-7; Bergers et al., 1999; Science; 284: 808-12; Klauber N et al. 1997; 57: 81-6). However, the mechanisms by which such agents exert their anti-tumor effect remain unclear (O'Reilly et al., 1997; Oreilly et al., 1996).

The interest in FGFs and their receptors as potential drug targets arose from their wide distribution in cells of different lineages and their mitogenic and angiogenic activities, two essential activities for solid tumor growth and metastasis (Zetter, B. R. 1998; Annu. Rev. Med. 49: 407-424). The oncogenic potential of FGFs is well documented in both, in-vitro studies and animal model systems (McKeehan, W. L., et al., 1998; Prog. Nucleic. Acid. Res. Mol. Biol. 59: 135-176; Szebenyi, G. and Fallon, J. F. 1999; Int. Rev. Cytol. 185: 45-106; Shaoul, E., et al., 1995; Oncogene 10: 1553-1561; Miki, T., et al., 1991; Science 251: 72-75; Kitsberg, D. I. and Leder, P. 1996. Oncogene 13: 2507-2515). Moreover, a variety of human carcinomas including pancreatic, endometrial and prostate carcinomas, overexpress FGFs (FGF-1, FGF-2 and FGF-7) and their receptors (KGFR and FGFR1), and tumor aggressiveness is correlated with the level of expression of these receptors (Giri, D., et al., 1999; Clin. Cancer Res. 5: 1063-1071; Visco, V., et al., 1999; Int. J. Oncol. 15: 431-435; Siegfried, S., et al., 1997; Cancer 79: 1166-1171; Ishiwata, T., et al., 1998; Am. J. Pathol. 153: 213-222; Kornmann, M., et al., 1998; Pancreas 17: 169-175; Siddiqi, I., et al., 1995; Biochem. Biophys. Res. Commun. 215: 309-315). Altogether, these observations strongly suggest the involvement of FGFs and their receptors in the malignant process. Thus, developing tools to target FGFRs, and their signaling pathways could be very useful for cancer therapy.

Several mechanisms collectively known as “negative signaling” have been evolved to attenuate signaling by RTKs (Christofori, G., 2003). One such mechanism involves ligand-induced antagonists of RTK signaling. The Sprouty and SPRED (Sprouty related EVH1-domain-containing) proteins belong to this category, and are regarded as general inhibitors of RTK signaling. They suppress the RTK-induced mitogen-activated protein kinase (MAPK) pathway (reviewed in Christofori, G., 2003; Dikic and Giordano, 2003).

Sef (for Similar Expression to FGF genes) is a newly identified antagonist of fibroblast growth factor (FGF) signaling. Sef encodes a putative type I transmembrane protein that is conserved across zebrafish, mouse and human but not in invertebrates (Furthauer, M., et al., 2002; Tsang M., et al., 2002; Lin, W., et al., 2002). Zebrafish Sef (zfSef) antagonizes FGF activity during embryogenesis by acting as a feedback-induced antagonist of the Ras/MAPK mediated FGF-signaling (Furthauer, M., et al., 2002; Tsang M., et al., 2002). Subsequent studies showed that the mouse (Kovalenko D, 2003) homologue of zfSef similarly inhibit FGF-induced activation of MAPK, and FGF-induced activation of protein-kinase B (pkB/Akt), a key protein in the phosphatidylinositol-3 (PI3) kinase pathway. On the other hand, the mSef was unable to inhibit PDGF-, EGF- or calf serum-induced phosphorylation of ERK in NIH 3T3 cells (Kovalenko D, 2003). Other studies showed that the human Sef homologue (which is later referred to as hSef-a by the present inventor), is capable of inhibiting FGF- and NGF-induced differentiation of PC12 cells (Xiong et al., 2003; JBC 278: 50273-50282).

While reducing the present invention to practice, the present inventor has uncovered three new alternatively spliced isoforms of the human Sef (designated hSefb-d) and demonstrated the capacity of hSefb to inhibit FGF and PDGF RTK signaling.

### SUMMARY OF THE INVENTION

According to one aspect of the present invention there is provided a method of inhibiting a growth of a solid tumor in a subject, the method comprising administering to the subject an agent capable of upregulating the expression level and/or activity of at least a functional portion of Sef, the at least a functional portion of Sef being capable of inhibiting RTK-mediated cell proliferation, thereby inhibiting the growth of the solid tumor in the subject.

According to another aspect of the present invention there is provided a pharmaceutical composition useful for inhibiting a growth of a solid tumor in a subject comprising, as an active ingredient, an agent capable of upregulating the expression level and/or activity of at least a functional portion of Sef, the at least a functional portion of Sef being capable of inhibiting RTK-mediated cell proliferation, and a pharmaceutically acceptable carrier.

According to further features in preferred embodiments of the invention described below, the RTK-mediated cell proliferation is ligand independent.

According to still further features in the described preferred embodiments the RTK-mediated cell proliferation is ligand-induced.

According to still further features in the described preferred embodiments the at least a functional portion of Sef is a polypeptide as set forth by SEQ ID NO:6.

According to still further features in the described preferred embodiments the at least a functional portion of Sef is a polypeptide as set forth by amino acid coordinates 1-10, 267-707 and/or 288-707 of SEQ ID NO:6.

According to still further features in the described preferred embodiments upregulating is effected by at least one approach selected from the group consisting of:

(a) expressing in cells of the subject an exogenous polynucleotide encoding at least a functional portion of Sef;

(b) increasing expression of endogenous Sef in cells of the subject;

(c) increasing endogenous Sef activity in cells of the subject; and

(d) introducing an exogenous peptide and/or exogenous polypeptide including at least a functional portion of Sef to the subject.

According to still further features in the described preferred embodiments the exogenous polynucleotide is a nucleic acid construct comprising a polynucleotide at least 90% identical to the polynucleotide sequence set forth in SEQ ID NO:4.

According to still further features in the described preferred embodiments the exogenous polynucleotide is a nucleic acid construct comprising a polynucleotide selected from the group consisting of SEQ ID NOs:4, 8, and 9.

According to still further features in the described preferred embodiments the nucleic acid construct further comprises a promoter capable of directing an expression of said polynucleotide in said cells of the subject.

According to still further features in the described preferred embodiments the promoter is selected from the group consisting of Cytomegalovirus (CMV) promoter, simian virus (SV)-40 early promoter, SV-40 late promoter, metallothionein promoter, murine mammary tumor virus promoter, Rous sarcoma virus (RSV) promoter, and

According to still further features in the described preferred embodiments the Sef is a polypeptide at least 90% homologous (identical+similar) to a polypeptide set forth by SEQ ID NO:6 as determined using the BlastP software where gap open penalty equals 11, gap extension penalty equals 1 and matrix is blosum 62.

According to still further features in the described preferred embodiments the Sef is a polypeptide set forth by SEQ ID NO:6.

According to still further features in the described preferred embodiments the solid tumor is selected from the group consisting of ovarian carcinoma, pancreatic cancer, breast cancer, endometrial carcinoma, brain tumor, adrenal carcinoma, pituitary cancer, thyroid carcinoma, tonsillar carcinoma, spleen cancer, adenoids cancer, kidney cancer, liver cancer, testis cancer, bladder cancer, colon cancer, prostate cancer, bile duct, lung cancer, and stomach cancer.

According to still further features in the described preferred embodiments the ligand is selected from the group consisting of FGF, PDGF, VEGF, NGF, insulin, and EGF.

According to still further features in the described preferred embodiments the RTK-mediated cell proliferation is effected by inhibition of activated Erk1/2 (P-Erk1/2) within said cells.

The present invention successfully addresses the shortcomings of the presently known configurations by providing agents and pharmaceutical compositions suitable for inhibiting the growth of solid tumors.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration depicting the structural homology between the human Sef-a and Sef-b isoforms. Shown are residues unique in each Sef isoform (bold letters), the signal for secretion (asterisk), potential N-linked glycosylation sites (arrows), transmembrane domain (black box), putative tyrosine phosphorylation site (Y), immunoglobulin like domain and the IL-17R-like domain (light and dark gray boxes). Note that while the human Sef-a contains a signal for secretion (the sequence appears left to the arrow), the hSef-b isoform lacks such a signal.

FIGS. 2a-b are autoradiographs depicting the identification of hSef protein products. FIG. 2a—Western blot analysis. HEK 293 cells were transiently transfected with either a control empty vector (lane 1) or Myc-tagged hSef-b or hSef-a vectors (lanes 2 and 3, respectively). Equal amounts of protein were subjected to SDS-PAGE followed by α-myc immunoblotting. FIG. 2b—in-vitro translation of hSef-b. In vitro transcription-translation was performed in the presence of [³⁵S]-methionine and either an expression vector including the hSef-b cDNA (lane 1) or an empty vector (lane 2). The translation products were analyzed by SDS-PAGE, and visualized by phospho-imaging.

FIGS. 3a-d are immunofluorescence staining depicting the cellular localization of the hSef isoforms. HEK 293 cells were transfected with the myc-tagged hSef-a (FIGS. 3a and b) or the myc-tagged hSef-b (FIGS. 3c and d) expression vectors, and 48 hours post-transfection the cells were subjected to immunostaining using α-myc (red; Figures a and c) or α-hSef (green; FIGS. 3b and d) antibodies. Nuclei were counterstained with bisbenzimide (blue).

FIGS. 4a-d are agarose gel images depicting the expression pattern of human Sef isoforms as determined by RT-PCR analyses. Total RNA from the indicated human tissues and human primary cells was subjected to RT-PCR using primers specific to the common hSef transcript (SEQ ID NOs:11 and 18, FIG. 4a), to the hSef-a (SEQ ID NOs:17 and 16, FIG. 4b), hSef-b (SEQ ID NOs:2 and 17, FIG. 4c), or GAPDH (SEQ ID NOs:12 and 13). Adrenal m=adrenal medulla; adrenal c=adrenal cortex; A.E. cells=primary aortic endothelial cells; F. Brain=fetal brain; A. Brain=adult brain; F. Kidney=fetal kidney; NC=negative control; PC=positive control. Templates for positive controls are plasmids containing hSef-a (FIGS. 4a-b); hSef-b (FIG. 4c) or GAPDH (FIG. 4d).

FIG. 5 is an autoradiograph depicting induced expression of hSef-b in the Tet-off NIH/3T3 cells. Cells were grown in 10% serum in the presence (lanes 1 and 3) or absence (lanes 2 or 4) of tetracycline. Following 24 hours, the cells were lysed and hSef-b expression was analyzed by immunoblotting with hSef specific antibodies. Lanes 1 and 2=Control cultures of parental cells transfected with an empty pTet-Splice vector; lanes 3 and 4=Cells transfected with the pTet-Splice-hSef-b vector.

FIGS. 6a-c are photomicrographs illustrating the effect of hSef-b on apoptosis. NIH/3T3/hSef-b cells were grown for 48 hours in the presence (FIG. 6a) or absence (FIGS. 6b and 6c) of tetracycline, and in the absence of tetracycline and serum (FIG. 6c). Cells were washed, fixed and apoptosis was then evaluated by TUNEL staining. Note the presence of apoptotic cells in the absence of tetracycline and serum (FIG. 6c).

FIGS. 7a-b are graphs illustrating the inhibition of the mitogenic activity of FGF2 by human Sef-b. Confluent cultures of control (FIG. 7a) or hSef-b expressing (FIG. 7b) cells were serum starved (in the presence of 0.2% serum) for 24 hours in the presence or absence of tetracycline. FGF2 was added at the above-indicated concentrations and [³H]-thymidine incorporation assay was performed as previously described (24, 26).

FIG. 8 is a Western Blot analysis illustrating the effect of hSef-b on cyclin D1 levels. Human Sef-b inducible NIH/3T3 cells were serum starved for 24 hours in the presence (lanes 1-5) or absence (lanes 6-10) of tetracycline, following which the cells were stimulated with FGF2 (20 ng/ml) for 0 (lanes 1 and 6), 5 (lanes 2 and 7), 8 (lanes 3 and 8), 12 (lanes 4 and 9), or 20 (lanes 5 and 10) hours. The levels of cyclin D1 were evaluated in total cell lysates over 20 hours of stimulation. Cyclin D1 and CDK proteins were analyzed by immunoblotting with anti-D1 monoclonal antibody and rabbit anti-CDK4, respectively. Note the presence of the Cyclin D1 protein in cells grown with tetracycline and the absence of such protein following tetracycline removal and activation of hSef-b expression.

FIGS. 9a-b are Western Blot analyses illustrating the effect of hSef-b on FGF2-induced signaling pathways. FIG. 9a—NIH 3T3 hSef-b expressing cells; FIG. 9b—NIH 3T3 cells transfected with an empty vector (control cultures). Equal amounts of total cells lysates were analyzed by immunoblotting. The membranes were successively incubated with the indicated antibodies. Lanes 1-4: cells were grown in the presence of tetracycline; lanes 5-8: cells were grown in the absence of tetracycline. Note that while in cells transfected with the control empty vector Erk1/2 activation was not influenced by tetracycline removal (FIG. 9b), in hSef-b-expressing cells tetracycline removal resulted in a decrease in the level of P-Erk1/2, but not the level of total Erk1/2. Each experiment was repeated at least twice and using two independent clones of hSef-b inducible cells. P-Erk 1/2, P-Akt, P-p38 and P-MEK1/2 are antibodies directed against the phosphorylated (P) form of each of the kinases.

FIGS. 10a-b are autoradiographs illustrating co-immunoprecipitation analyses of Sef isoforms and FGFR1. HEK 293 cells were transfected with the indicated constructs and immunoblotting with α-FGFR1 (H76) or α-hSef antibodies was performed on whole cell lysates (WCL; FIG. 10b) or on cell lysates following α-myc immunoprecipitation (IP; FIG. 10a). Lane 1—transfection with the FGFR1 construct; lane 2—transfection with hSef-a construct containing the c-myc epitop at the C-terminus (hSef-a::myc); lane 3—co-transfection with the FGFR1 and the hSef-a::myc constructs; lane 4=transfection with the hSef-b construct containing the c-myc epitop at the C-terminus (hSef-b::myc); lane 5—co-transfection with the FGFR1 and the hSef-b::myc constructs.

FIGS. 11a-b are bar graphs depicting mitogenic assays in control (FIG. 11a) or hSef-b expressing (FIG. 11b) cells. Confluent cultures were subjected to mitogenic assays as describe in FIGS. 7a-b and the fold increase (FI) in biological activity was calculated by dividing CPM values obtained in the presence of the indicated stimulators with those obtained in the presence of 0.2% serum alone. Percent [³H]-thymidine incorporation is relative to FI obtained in cultures stimulated with 10% serum in the presence of tetracycline that was set as 100%. The concentrations of FGFs, insulin, EGF and serum are those which gave rise to a maximal biological response. F=FGF; INS=insulin.

FIG. 12 is a Western Blot analysis illustrating the effect of hSef-b-mediated PDGF inhibition of ERK1/2 MAPK level. Human Sef-b inducible NIH/3T3 cells were serum starved for 24 hours in the presence (lanes 1-3) or absence (lanes 4-6) of tetracycline, following which the cells were stimulated with 20 ng/ml PDGF for the indicated time periods. Equal amounts of total cells lysates were analyzed by immunoblotting using anti-P-Erk 1/2 and anti-ERK antibodies. These experiments were repeated at least 3 times and using two independent hSef-b expressing clones.

FIGS. 13a-b depict the nucleic acid (FIG. 13a) and amino acid (FIG. 13b) sequences of hSef-c as determined using the RACE products. Nucleic acids and amino acids from the unique hSef-c domain are labelled in red. The potential initiation Methionine codons are labelled in green. *=termination codon.

FIGS. 14a-b depict the nucleic acid (FIG. 14a) and amino acid (FIG. 14b) sequences of hSef-d as determined using the RACE products. Nucleic acids and amino acids from the unique hSef-d domain are labelled in red. The potential initiation Methionine codon is labelled in green.

### DESCRIPTION OF THE PREFERRED EMBODIMENTS

The present invention is a method of inhibiting solid tumor growth using an agent capable of upregulating the expression level and/or activity of hSef-b in FGF and/or PDGF-induced proliferating cancerous cells, and tumors that overexpress their cognate receptors. Specifically, the present invention can be used to treat cancerous tumors such as ovarian carcinoma, pancreatic cancer, breast cancer, endometrial carcinoma and brain tumors.

The principles and operation of the method of inhibiting solid tumor growth according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Solid tumors account for the majority of human tumors and among them, carcinomas of an epithelial origin, account for over 80%. Conventional cancer therapy is based on chemotherapy drugs (e.g., thymidylate synthase inhibitors, nucleoside analogs, aromatase inhibitors, taxanes and topoisomerase-I inhibitors) which often fail due to the development of drug resistance by the cancerous cells.

Since the growth of solid tumors depends on nutrients and oxygen which are supplied by the tumor vasculature, new approaches of cancer therapy are focused on the inhibition of angiogenesis via the inhibition of receptor tyrosine kinases and their ligands. Thus, various anti-angiogenic agents have been designed and tested in animal models (Boehm et al., 1997; Nature; 390: 404-7; Bergers et al., 1999; Science; 284: 808-12; Klauber N et al. 1997; 57: 81-6). However, the mechanisms by which such agents exert their anti-tumor effect remain unclear (O'Reilly et al., 1997; Oreilly et al., 1996).

Recently, new ligand-induced antagonists of RTK signaling have been discovered. These include the Sprouty and SPRED (Sprouty related EVH1-domain-containing) proteins which suppress the RTK-induced mitogen-activated protein kinase (MAPK) pathway (reviewed in Christofori, G., 2003; Dikic and Giordano, 2003).

Sef (for Similar Expression to FGF genes) is a newly identified antagonist of fibroblast growth factor (FGF) signaling which antagonizes FGF activity during embryogenesis by acting as a feedback-induced antagonist of the Ras/MAPK mediated FGF-signaling (Furthauer, M., et al., 2002; Tsang M., et al., 2002). Sef encodes a putative type I transmembrane protein that is conserved across zebrafish, mouse and human (Yang R B, et al., 2003 and data not shown) but not in invertebrates (Furthauer, M., et al., 2002; Tsang M., et al., 2002; Lin, W., et al., 2002). Subsequent studies showed that the mouse (Kovalenko D, 2003) homologue of zfSef similarly inhibits FGF-induced activation of MAPK, and FGF-induced activation of protein-kinase B (pkB/Akt), a key protein in the phosphatidylinositol-3 (PI3) kinase pathway. On the other hand, the mSef was unable to inhibit PDGF-, EGF- or calf serum-induced phosphorylation of ERK in NIH 3T3 cells (Kovalenko D, 2003). Other studies showed that the human Sef homologue (which is later referred to as hSef-a by the present inventor), is capable of inhibiting FGF- and NGF-induced differentiation of PC12 cells (Xiong et al., 2003; JBC 278: 50273-50282).

FGFs comprise a family of 22 structurally related polypeptide mitogens that control cell proliferation, differentiation, survival and migration, and play a key role in embryonic patterning (14-16). They signal via binding and activation of a family of cell surface tyrosine kinase receptors designated FGFR1-FGFR4 (17-20). Activated receptors trigger several signal transduction cascades including the Ras/MAPK and the PI3-kinase pathway (15, 21). Depending on the cell type, FGF can also activate other MAPK pathways, such that leading to the activation of p38-MAPK (22, 23).

PDGF acts as a potent mitogen of mesenchymal cell proliferation via the two related receptor tyrosine kinases, alpha and beta PDGF receptors and its expression was demonstrated in various solid tumors, such as glioblastomas and prostate carcinomas (Reviewed in George D. 2003; Adv Exp Med Biol. 532:141-51).

While reducing the present invention to practice, the present inventor has uncovered three new alternatively spliced isoforms of the human Sef (designated hSefb-d) and demonstrated that hSef-b is uniquely capable of inhibiting FGF and PDGF RTK signaling, inhibiting the growth of cells expressing FGF and PDGF cognate receptors and reducing solid tumor growth.

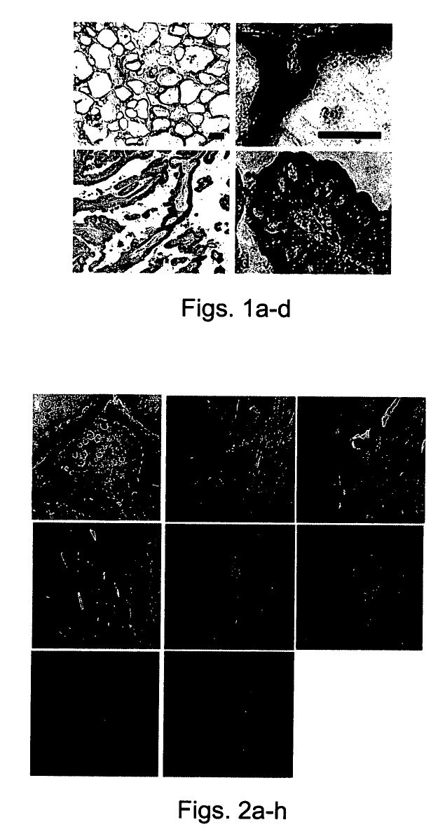

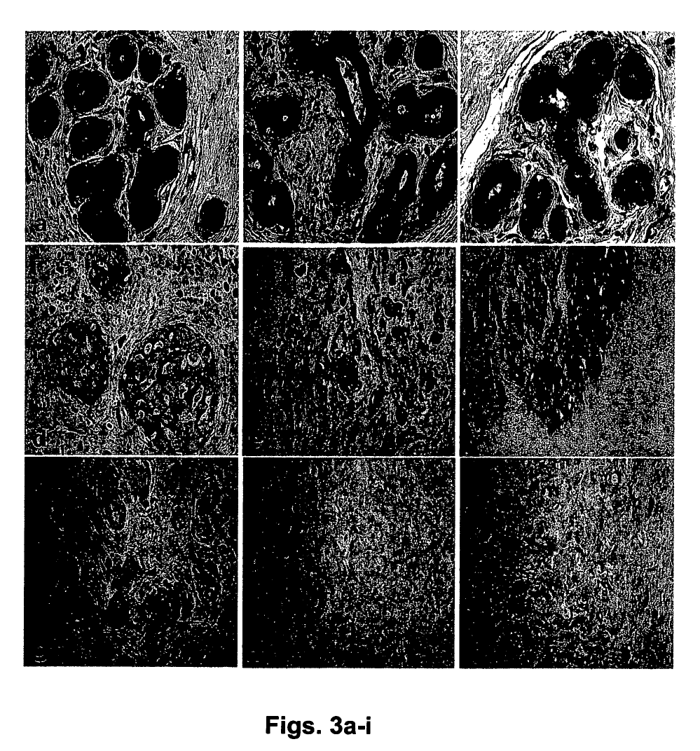

As is shown in FIGS. 1-4 and Examples 1 and 2 of the Examples section which follows, while the hSef-a protein is a heavily glycosylated membrane protein, the hSef-b protein is a cytosolic protein, lacking post-translational glycosylations. Furthermore, the cytosolic isoform of the human Sef protein (hSef-b), but not the transmembrane isoform (hSef-a, See Lin W., et al., 2002) is capable of inhibiting the growth of NIH/3T3 cells via the inhibition of FGF and PDGF signaling pathway (FIGS. 1-2 and 4-12, Table 1 and Examples 1, 3, 4, and 5 of the Examples section which follows). In addition, as is shown in FIGS. 2a-b and Example 1 of the Examples section which follows, hSef-b is translated from a Leucine initiation codon instead of a Methionine codon. Such a usage of an alternative translation initiation codon is probably the reason for the relatively low levels of hSef-b obtained following transfection (data not shown). Notwithstanding, hSef-b was found to be more potent in inhibiting RTK-mediated cell proliferation than hSef-a (data not shown). Altogether, these results demonstrate that the unique N-terminal sequence of hSef-b (amino acids 1-10 in SEQ ID NO:6) and/or the lack of secretion signal and/or the intracellular location of hSef-b confer upon this protein (hSef-b) unique properties as a potent inhibitor of RTK-mediated cell proliferation with a wide repertoire of substrates and/or interacting proteins.

Thus, according to one aspect of the present invention there is provided a method of inhibiting a growth of a solid tumor in a subject.

As used herein, the phrase “inhibiting a growth” refers to arresting the development, causing the reduction, remission, or regression of a solid tumor and/or eradicating tumor cells. Those of skill in the art will be aware of various methodologies and assays which can be used to assess the development of a solid tumor (e.g., biopsy or fine needle aspiration followed by histopathological examination, ultrasound scan, C.T. scan, X-ray, NMR and the like), and similarly, various methodologies and assays which can be used to assess the reduction, remission or regression of the solid tumor.

The phrase “solid tumor” as used herein, refers to an abnormal mass of tissue resulting from excessive cell division. Solid tumors can be benign or cancerous (i.e., with unregulated proliferation). Preferably, the phrase “solid tumor” as used herein relates to an unregulated cancerous tumor including, but not limited to, ovarian carcinoma, pancreatic cancer, breast cancer, endometrial carcinoma, brain tumor, adrenal carcinoma, pituitary cancer, thyroid carcinoma, tonsillar carcinoma, spleen cancer, adenoids cancer, kidney cancer, liver cancer, testis cancer, bladder cancer, colon cancer, prostate cancer, bile duct, lung cancer, stomach cancer, and the like.

The method is effected by administering to the subject an agent capable of upregulating the expression level and/or activity of at least a functional portion of Sef, such a functional portion of Sef being capable of inhibiting RTK-mediated cell proliferation, thereby inhibiting the growth of the solid tumor in the subject.

As used herein, the term “subject” refers to a mammal, preferably a human being (male or female) at any age which is diagnosed with a cancerous solid tumor as described hereinabove or is predisposed to developing such a tumor.

According to preferred embodiments of the present invention the functional portion of Sef encompasses a Sef protein derived sequence (i.e., of any of the hSef isoforms and homologues described herein, preferably hSef-b) which is functional as an RTK receptor antagonist and thus is capable of inhibiting RTK-mediated cell proliferation via the inhibition of activated ERK1/2.

It will be appreciated that since hSef-b (SEQ ID NO:6) lacks the secretion signal and is found entirely in the cytosol, its entire coding sequence (i.e., amino acid 1-707 in SEQ ID NO:6) can potentially contribute to its functional activity (i.e., inhibiting RTK mediated cell proliferation).

Thus, according to preferred embodiments of the present invention the functional portion of Sef is encompassed by the sequence set forth by amino acids 1-707 of SEQ ID NO:6.

Since hSef-a and hSef-b share a large common domain (i.e., amino acids 11-707 of SEQ ID NO:6) of which a large portion is cytosolic [i.e., amino acids 320-739 in SEQ ID NO:5 (hSef-a) which are identical to amino acids 288-707 in SEQ ID NO:6 (hSef-b)], the functional portion of Sef is preferably encompassed by amino acids 288-707 of SEQ ID NO:6.

Additionally or alternatively, based on targeted deletions made using hSef-a DNA constructs (Xiong S., et al., 2003, JBC 278: 50273-50282; Torii S., et al., 2004, Developmental Cell 7:33-44) which demonstrated the presence of domains required for hSef-a activity, it is conceivable that the equivalent sequence in hSef-b would contribute to the functional activity of hSef-b, i.e., inhibiting RTK-mediated cell proliferation. Thus, the functional portion of Sef is preferably encompassed by amino acids 267-707 of SEQ ID NO:6.

Yet additionally or alternatively, since as is mentioned above hSef-b is a more potent inhibitor of RTK-mediated cell proliferation than hSef-a, it is conceivable that the unique sequence of hSef-b (i.e., amino acids 1-10 in SEQ ID NO:6) is likely to contribute to the functional activity of Sef. Thus, according to preferred embodiments of the present invention the functional portion of Sef includes at least amino acids 1-10 of SEQ ID NO:6, more preferably, at least amino acids 1-10 and 267-707 of SEQ ID NO:6, more preferably, at least amino acids 1-10 and 288-707 of SEQ ID NO:6.

It will be appreciated that the method of the present invention can also utilize Sef homologues (identified from other species or other hSef isoforms) which exhibit the above described functional activity.

Preferably, the polypeptide used by the present invention is at least 85%, at least 86%, at least 87%, at least 88%, at least 89%, at least 90%, at least 91%, at least 92%, at least 93%, at least 94%, at least 95%, at least 96%, at least 97%, at least 98%, most preferably, at least 99% homologous (similar+identical) to the polypeptide sequence set forth in SEQ ID NO:6 or to a portion thereof, as determined using the BlastP software available from the NCBI (http://www.ncbi.nlm.nih.gov) where gap open penalty equals 11, gap extension penalty equals 1 and matrix is blosum 62.

As used herein, the phrase “RTK-mediated cell proliferation” refers to RTK activation, signaling and/or over-expression which can be either ligand-induced or ligand independent. Non-limiting examples of RTK ligands include FGF, PDGF, NGF, VEGF, insulin, PLGF, c-kit, met and EGF. Ligand independent RTK-mediated cell proliferation is often found in cancerous tumors such as neuroblastoma [e.g., TrkA (Gryz E A and Meakin S O, 2003; Oncogene. 22: 8774-85)], gliomas (Kapoor G S and O'Rourke D M, 2003, Cancer Biol. Ther. 2: 330-42), lung cancer [e.g., c-Met (Ma P C, et al., 2003; Cancer Res. 63: 6272-81)], breast and prostate cancers [e.g., HER2 (Witton C J et al., 2003, J Pathol. 200: 290-7)].

As is mentioned hereinabove, the present invention preferably targets the RTKs ligands FGF and PDGF. It will be appreciated that activity of other RTK ligands which are capable of inducing cell proliferation via the activation of ERK1/2, can also be inhibited by the various hSef isoforms of the present invention.

It is also highly likely that unique sequences present in splice variants hSef-c (i.e., amino acids 8-49, 9-49 or 28-49 of SEQ ID NO:15) and hSef-d (i.e., amino acids 37-50 of SEQ ID NO:14) are involved in specific RTK-mediated cell proliferation and/or signaling and as such, the present invention also envisages using these sequences in the method of the present invention.

As is mentioned hereinabove, the present method is effected by upregulating the expression level and/or activity of at least a functional portion of Sef.

The term “upregulating” relates to increasing the expression and/or activity of Sef.

Upregulation of Sef can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like). For example, upregulation can be effected using hormones or their agonists which upregulate Sef transcription or stabilize Sef RNA transcripts as is further described hereinbelow.

Following is a list of agents capable of upregulating the expression level and/or activity of Sef.

An agent capable of upregulating expression level of a Sef may be an exogenous polynucleotide sequence designed and constructed to express at least a functional portion of the Sef protein. Accordingly, the exogenous polynucleotide sequence may be a DNA or RNA sequence encoding a Sef molecule, capable of inhibiting RTK ligand-induced cell proliferation.

The entire coding region of Sef has been cloned from Zebrafish, mouse, human and chicken and partial cDNA clones are also available from bovine. Thus, coding sequences information for Sef is available from several databases including the GenBank database available through http://www.ncbi.nlm.nih.gov/.

To express exogenous Sef in mammalian cells, a polynucleotide sequence encoding Sef (SEQ ID NO:6) or a functional portion of Sef (amino acids 1-707 of SEQ ID NO:6 or a portion thereof) is preferably ligated into a nucleic acid construct suitable for mammalian cell expression. Such a nucleic acid construct includes a promoter sequence for directing transcription of the polynucleotide sequence in the cell in a constitutive or inducible manner.

It will be appreciated that the nucleic acid construct of the present invention can also utilize Sef homologues encoding polypeptides which exhibit the desired activity (i.e., inhibiting RTK-mediated cell proliferation). Such homologues can be, for example, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO:4 or a portion thereof, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tet off system (Shockett, P., Difilippantonio, M., Hellman, N. & Schatz, D. G. (1995) Proc. Natl. Acad. Sci. U.S.A. 92: 6522-6526).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of Sef mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, PLNCX (a virus shuttle vector), pAV009/A⁺, pMTO10/A⁺, pMAMneo-5, baculovirus pDSVE, and any other vector allowing expression of proteins under the direction of the SV-40 early promoter, SV-40 late promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, kidney cells may be targeted using the heterologous promoter present in the baculovirus Autographa californica nucleopolyhedrovirus (AcMNPV) as described in Liang C Y et al., 2004 (Arch Virol. 149: 51-60) and ovarian cancer cells may be targeted using a recombinant adeno-associated virus-2 (rAAV) as described by Mahendra G, et al. (Cancer Gene Ther. 2004 Sep. 10 [Epub ahead of print].

Recombinant viral vectors are useful for in vivo expression of Sef since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into the targeted cells (i.e., the cancerous cells of the solid tumor). Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

An agent capable of upregulating Sef may also be any compound which is capable of increasing the transcription and/or translation of an endogenous DNA or mRNA encoding the Sef polypeptide and thus increasing endogenous Sef activity. For example, FGF was found to increase Sef transcripts in chick embryos (data not shown and Ron D., ASBMB annual meeting, Boston, Jun. 12-16, 2004). In addition, as determined by RT-PCR analysis, various hormones such as FSH and LH-like hormones are capable of upregulating Sef transcription in mice ovary (data not shown). It will be appreciated that many other hormones or growth factors such as androgens, TSH, PDGF, NGF, insulin, and/or VEGF are highly likely to upregulate Sef transcription, RNA stability, translation and/or activity.

Thus, according to preferred embodiments of the present invention, the upregulating agent used by the present invention a growth factor and/or a regulating hormone.

An agent capable of upregulating Sef may also be an exogenous polypeptide including at least a functional portion (as described hereinabove) of the Sef protein. Methods of identifying such exogenous polypeptides are known in the art (Torii S, et al., 2004; Developmental Cell 7: 33-44; Tsang et al., 2002). Upregulation of Sef can be also achieved by introducing to the subject at least one Sef substrate. Non-limiting examples of such agents include hormones such as FSH and LH which can be administered orally, intravenously or locally directly to the tumor tissue to thereby upregulate Sef expression level and/or activity within specific tumor cells.

It will be appreciated that since FGFR is expressed in many types of tumors, FGF and Sef (or an expression vector encoding same) can be co-administered to the subject to facilitate and/or improve Sef activity in reducing and/or inhibiting tumor growth.

It will be appreciated that the differential expression of the various hSef isoforms (see Examples 2 and 6 of the Examples section which follows) may suggest the use of tissue-specific Sef isoforms for the inhibition of tissue-specific tumor growth. For example, upregulation of hSef-b in a tissue where it is usually not expressed (e.g., adrenal or ovary) or moderately expressed (e.g., testes) can lead to efficient inhibition of tumor growth and subsequent treatment of the cancer.

Each of the upregulating agents described hereinabove or the expression vector encoding Sef or a functional portion thereof can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the upregulating agent or the expression vector encoding Sef or a functional portion thereof which are accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (the upregulating agent or the expression vector encoding Sef) effective to prevent, alleviate or ameliorate symptoms of a disorder (e.g., inhibit tumor growth) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (See e.g., Fingl, et al., 1975, in “The Pharmacological Basis of Therapeutics”, Ch. 1 p. 1).

Dosage amount and interval may be adjusted individually to provide plasma levels of the active ingredient are sufficient to inhibit tumor growth (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Thus, the teachings of the present invention can be used to treat individuals having a cancerous tumor (e.g., a solid tumor in the kidney). For example, an expression vector (e.g., a viral vector) including a polynucleotide sequence encoding the human Sef-b mRNA (SEQ ID NO:4) and the suitable promoter sequences to enable expression in kidney cells is introduced into the individual via intravenous administration. Expression of such a vector in the kidney is expected to upregulate the expression level and/or activity of Sef in this tissue and thus to inhibit FGF and/or PDGF-induced cell proliferation in the tumor cells. Dosage of such an expression vector should be calibrated using cell culture experiments and animal models. Success of treatment is preferably evaluated by determining the tumor size (using for example C.T. scans) and the individual general health status.

It will be appreciated, that if such a treatment is employed early following the detection of a cancerous tumor, it may prevent the complications associated with tumor growth (e.g., metastases) and thus improve the individual's prognosis quality of life.

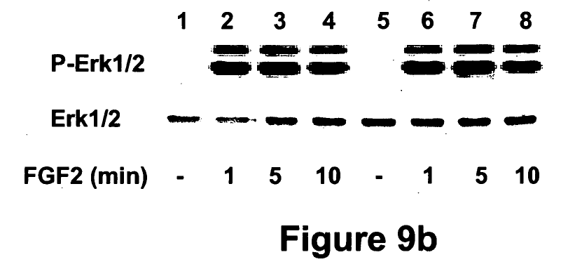

It will be appreciated that the agent of the present invention (which increases the transcription, translation and/or activity of Sef), can be identified using a variety of methods known in the art. For example, cells expressing low levels of endogenous Sef (e.g., NIH 3T3 cells) can be cultured in 10-cm dishes in the presence of a culture medium (e.g., DMEM medium) supplemented with serum (e.g., 10% newborn calf serum). To identify a potential agent which upregulates the transcription, translation and/or activity of Sef, various transcription factors, drugs and molecules such as hormones (e.g., FSH and LH-like hormones) can be added to the culture medium for an incubation period which is expected to cause upregulation of transcription, translation and activity. As is shown in FIGS. 9a-b, such incubation period can vary between minutes to hours, and it is within the capabilities of those skilled in the art to determine the suitable incubation periods.

Since the functional portion of Sef is included in the intracellular fraction (see Example 1 of the Examples section which follows), the effect of the agent on Sef expression levels and/or activity of Sef is preferably determined by analyzing the cells and not the cell medium. Thus, the cells are collected and centrifuged, the medium is discarded and the cell pellet is further subjected to RNA and/or protein detection methods.

Alternatively, the anti-mitogenic activity of Sef (see Example 3 of the Examples section which follows) can be measured by determining the level of DNA synthesis in such cells (a mitogenic assay), or counting cell number as a measure for Sef effect on proliferation.

Following is a list of methods useful for detecting Sef RNA level in cells.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers [e.g., the forward and reverse hSef-b primers (SEQ ID NOs:2 and 1, respectively)] and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The probe can be for example an RNA-oligonucleotide probe (e.g., a 5′-biotinylated 2-O-methyl RNA oligonucleotide) which is synthesized according to a selected sequence from the unique 5′ region of hSef-b (i.e., nucleic acids 1-240 as set forth in SEQ ID NO:7). The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Following is a list of immunological detection methods which can be used to detect the level of Sef protein in cells.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis. In addition, using phospho-specific antibodies, this method allows the determination of the activation state of specific proteins [e.g., P-p38 or P-ERK1/2 (see Example 4 of the Examples section which follows)].

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate (i.e., Sef) in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

It will be appreciated that Sef activity (i.e., inhibition of RTK ligand-induced proliferation and/or downregulation of the over-expressed RTK receptors via the inhibition of P-ERK1/2) can be detected as described in details in Examples 3-5 of the Examples section which follows.

Thus, the agent of which addition to the cells results in upregulation of the expression level and/or activity of endogenous Sef in the cells is identified as a potential upregulating agent and can be used to inhibit tumor growth in the subject.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N. Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Material and Experimental Methods

Enzymes, growth factors, reagents and chemicals—Restriction enzymes and Taq-polymerases were from NEB (Beverly, Mass.), Pharmacia (Amersham Pharmacia Biotech LTD, UK) and Roche (Indianapolis, Ind., USA). Purified recombinant FGF2 was produced as described (24-26). Bovine brain FGF1, recombinant human FGF4, epidermal growth factor (EGF) and platelet derived growth factor (PDGF) were from R&D Systems (R&D Systems Inc, Minneapolis, Minn., USA). [³⁵S]-Methionine (1000 Ci/mmol) and [³H]-Thymidine (25 Ci/mmole) were from Amersham HVD Biotech. Fibronectin, fetal and newborn calf serum and media were from Biological Industries (Beth Ha'emek, Israel, Israel) or Gibco-Laboratories (Grand Island, N.Y., USA). Fluoromount-GTM was from Southern Biotechnology Associates, Inc. (Birmingham, Ala., USA). BSA was from ICN (UK). All other chemicals were from Sigma (St Louis, Mo.).

cDNA cloning and plasmid construction—RT-PCR was utilized to amplify the entire coding region of hSef-a from human brain or fibroblast RNA, and of hSef-b from testes RNA. First strand cDNA was synthesized using the following primer: 5′-AGTGGCAATGCTTAGACTCTTTCGT-3′ (SEQ ID NO:1) which is designed according to the 3′ un-translated region (UTR) of a partial hSef EST clone (GenBank Accession No. AL133097, i.e., a common primer for both hSef-a and b isoforms). Amplification of the coding region of hSef-b was performed with nested primer: 5′-GAGGATCCAAGCTTTGTTACAAAGGGGCGACCGCGT-3′ (SEQ ID NO:3), and a primer flanking the amino terminal part unique to the hSef-b isoform: 5′-GCGTGCCAGACAGAGTGCTAGGCAT-3′ (SEQ ID NO:2) which is designed according to the Testes EST clone GenBank Accession No. BG721995 and is located at the unique 5′ sequence of hSef-b (nucleic acids 126-150 of SEQ ID NO:7; This clone was served as a platform to reconstruct hSef-a cDNA containing its entire ORF. Thus, the 5′ portion of hSef-a was amplified from human brain or normal fibroblasts using a primer unique to hSef-a: 5′-GAGGATCCTGACGGCCATGGC CCCGTGGCTGCAGCTC-3′ (SEQ ID NO: 16, which is designed according to the EST clone GenBank Accession No. BE750478 and is located at the unique 5′ sequence of hSef-a (nucleic acids 1-37 of SEQ ID NO:10), and a primer common to both Sef isoforms (SEQ ID NO:3). This fragment was digested with BamH1 following sequence verification and ligated to the remainder of hSef-a sequences. The cDNA of hSef-a or hSef-b was cloned into pcDNA3.1, pTET splice and pcDNA3.1/myc-His expression vectors (Invitrogen).

Analysis of the expression pattern of hSef transcripts—Total RNA was extracted from human tissues and cell lines as described elsewhere (19). Two μg of total RNA were used for first strand synthesis with random hexamer primers. RT-PCR was performed with primers specific to the common region (SEQ ID NO:11 and SEQ ID NO:18); the hSef-a (SEQ ID NO:16 and SEQ ID NO:17); the hSef-b (SEQ ID NO:2 and SEQ ID NO:17); the hSef-c (SEQ ID NO:20 and SEQ ID NO:19); and the GAPDH transcript (SEQ ID NO:12 and SEQ ID NO:13). Amplification was performed in a solution of 1 μM of each oligonucleotide primer, 0.2 mM of each dNTPs, 50 mM KCl, 2 mM MgCl₂, 1 mM β-mercoptoethanol, 25 mM TAPS pH 9.3 and 0.02 units of Super-Therm polymerase in a total volume of 25 μl. After pre-denaturation at 95° C. for 5 min, the reaction mixture was incubated as follows: 30 sec at 94° C., 30 sec at 65° C., and 2 min at 72° C. for 25 (GAPDH), 35 (hSef-a and hSef-b) or 40 (hSef-c) cycles. 5 μl of each reaction were analyzed by gel electrophoresis on 1.8% agarose gels in TAE buffer (40 mM Tris-acetate, 1 mM EDTA) and stained with ethidium bromide. The PCR products of the amplification with hSef-c specific primers were separated by electrophoresis on 3% Nusieve-agarose gels (3% Nusieve GTC agarose, 1% Seakem LE agarose in TBE buffer) in TBE buffer (45 mM Tris-Borate, 1 mM EDTA).

Cell Cultures—HEK 293 and NIH/3T3 cells were grown in Dulbecco's modified Eagle's medium (DMEM) containing 10% fetal bovine serum or newborn calf serum, respectively.

Transient transfections of HEK 293 cells—Transient transfections of HEK 293 cells were performed using Lipofectamine Plus in Opti-MEM (Invitrogen). For co-immunoprecipitation (CO-IP) assays the cells were transiently transfected with hSef-a or hSef-b in the presence or absence of FGFR1. Briefly, cells were cultured in 6-cm dishes and were transfected with 3 μg of each FGFR1 and hSef-a plasmids (for hSef-a/FGFR1 CO-IP) or cells (in 10-cm dishes) were transfected with 4 μg of FGFR1 and 12 μg of hSef-b plasmids (for hSef-b/FGFR1 CO-IP).

Stable transfections in NIH/3T3 cells—Stable transfections were performed using calcium-phosphate, essentially as described in Ron, D. et al., 1988 (27). Tet-off NIH3T3 cells [S2-6 cells, a gift from Dr. David. G. Schatz (28)] were cultured in histidine-deficient DMEM containing 0.5 mM L-histidinol, serum, and 1 μg/ml tetracycline (tet). HSef-b Tet-off NIH/3T3 cell lines were established by co-transfection of the S2-6 cells with pTet splice-hSef-b or the same vector without the insert (i.e., an empty vector) and pTK-Hyg (Clontech) followed by selection in complete medium plus 150 μg/ml hygromycin. Colonies of resistant cells were isolated 3 weeks post transfection.

Cell Growth and Apoptosis Assays—[³H]-thymidine incorporation assay was done in 96-well microtiter plates as described elsewhere (24, 26). Confluent cultures were growth arrested in 0.3% serum for 24 hours, and when indicated, tet was removed 24 hours prior to serum (10%) or growth factors stimulation. For apoptosis studies, the control cells (S2-6) or S2-6/hSef-b cells were grown for 48 hours in the presence or absence of tet. The level of apoptosis was detected using the In Situ Cell Death detection kit (TUNEL staining, Roche Molecular Biochemicals) followed by a confocal microscopy examination.

In vitro translation of the hSef-b construct (pcDNA3.1/Hygro-hSef-b)—In vitro translation of hSef-b was performed using TnT Quick Coupled Transcription/Translation System in the presence of [³⁵S]-methionine according to manufacturer's instructions (Promega). Translation products were analyzed by SDS-PAGE and visualized using Phospho-imaging.

hSef antibodies—Polyclonal antibodies against hSef were generated by injecting rabbits with a polypeptide containing the last 402 residues of hSef fused to the amino-terminal portion of Bactriophage a T7 θ10 protein as described in Studier, F W et al., 1990 (29).

CO-IP and immunoblotting—were performed essentially as described elsewhere (30). Briefly, for CO-IP, cells were lysed 24 hours post transfection in HTNG buffer (20 mM HEPES, pH 7.4, 150 mM NaCl, 10% glycerol, 1% Triton X-100, 1 mM EGTA, 1 mM NaVO₄ and protease inhibitors). After incubation with antibody, immunocomplexes were captured on Protein G Dynabeads (Dynal), and washed with HTNG buffer. Following SDS-PAGE and immunoblotting, bound antibodies were visualized by chemiluminescence.

Immunofluorescence analysis—Transfected HEK 293 cells were fixed 48 hours post transfection with 4% paraformaldehyde, and incubated in PBS containing 1% BSA, 0.1% saponin and were subjected to immunostaining using the anti-myc (α-myc) antibody (anti-myc epitope, 9E10, Santa Cruz, Santa Cruz, Calif.) or the anti-human Sef antibody (α-hSef). Nuclear staining was done using 10 μM DRAQ5 (Biostatus limited, Shepshed, Leicestershire, UK). Examination of the immunofluorescent staining was performed using the MRC-1024 laser confocal microscope (BioRad, Hercules, Calif.).

Antibodies used in Western Blot and immunoprecipitation analysis—Commercial antibodies were obtained from various suppliers. The anti-myc epitope (9E10), p38, MEK, Akt, CDK4 and FGFR1 (H-76) rabbit polyclonal antibodies were from Santa Cruz; the phospho-p44/42 MAP kinase [Thr202/Tyr204], E10 mouse monoclonal antibodies (NEB), Phospho-Akt [Ser473], phospho-p38, peroxidase conjugated-goat anti-rabbit or anti-mouse IgG were from Sigma; the cyclin D1 and phospho-MEK were from Cell Signaling (Arundel, Australia); the FITC conjugated-Goat anti-rabbit IgG was from ICN; the Rhodamine-Red™-x-conjugated Affinipure goat anti-mouse IgG was from Jackson Immunoresearch (West Grove, Pa., USA). The ERK-2 rabbit polyclonal antibodies was a gift from Dr. Y. Granot, Ben Gurion University, Israel.

### Example 1

### Cloning of the Human Sef-b Isoform

Experimental Results

Database search revealed the presence of additional human Sef isoforms—A database search with the zfSef sequence revealed an expressed sequence tag (EST clone AL133097) containing the entire 3′-UTR of hSef and most of its coding region except for the first 170 residues. The remainder of the coding region was obtained by searching the human genome database for upstream exons of hSef and based on homology with bovine Sef (EST clone BE750478). A cDNA fragment encoding the entire open reading frame (ORF) of hSef was amplified from primary human fibroblast or human fetal brain RNA (FIG. 1a and data not shown). Human Sef has been mapped to a single locus on chromosome 3p14.3 (data not shown). Further database searches with the amino-terminal sequence of hSef revealed an EST clone from human testes that was 577 nucleotides long and contained an ORF of 122 residues. This EST clone differed from the original hSef in its amino-terminal and the upstream 5′-UTR sequences. Since the human genome contains a single Sef locus, these findings suggest the existence of alternatively spliced Sef isoforms.

Cloning of the human Sef-b isoform—To examine the possibility of additional hSef isoforms, an RT-PCR analysis was performed as follows: first strand was synthesized using a complementary to the 3′ UTR of hSef (SEQ ID NO:1), Amplification was performed with primers set forth by SEQ ID NOs:3 and 2. A single product of 2214 nucleotides was obtained (SEQ ID NO:4), confirming the existence of alternate isoforms of hSef (designated hSef-a and hSef-b for the brain and testes isoforms, respectively).

Identification of the hSef-b using RACE analysis—The present inventor also identified the hSef-b common and unique sequences using the RACE protocol on human keratinocytes RNA with three consecutive primers designed according to the common region (e.g., SEQ ID NO:17). The experimentally sequence obtained using the RACE protocol is provided in SEQ ID NO:22.

Human Sef-b isoform contains alternative N-terminal amino acid residues—As is schematically illustrated in FIG. 1, while the human Sef-a isoforms contains an ORF of 739 residues (SEQ ID NO:5), the human Sef-b contains an ORF of 707 amino acid residues (SEQ ID NO:6), of them the last 697 residues (i.e., amino acids 11-707 of SEQ ID NO:6) are identical in both isoforms. These results demonstrate the presence of alternative splicing in the human Sef gene resulting in the substitution of the first 42 amino acid residues of hSef-a ORF with 10 new residues of the hSef-b ORF.

Human Sef-b isoform lacks a secretion signal—Similar to hSef-a, the new isoform contains 8 potential N-linked glycosylation sites, a potential transmembrane spanning domain and tyrosine phosphorylation site, immunoglobulin and IL-17 receptor like domains (FIG. 1). Unlike hSef-a which contains a signal for secretion [amino acid residues 1-34 as set forth by SEQ ID NO:5; FIG. 1, marked by a star (*) and the sequence which appears left to the arrow], the hSef-b isoform lacks such a signal (FIG. 1), suggesting that it is not a secreted or trans-membrane protein.

Human Sef-b isoforms contains a putative CUG translation initiation codon Interestingly, the unique hSef-b region exhibits a CUG initiation codon. The next putative initiation codon (AUG) is located within the region that is identical in both isoforms. Thus, while translation from the CUG codon is expected to result in a protein of 707 amino acids with a predicted molecular weight of 78 kDa, translation from the AUG codon is expected to result in a protein of 595 residues and a predicted molecular weight of 65 kDa.

The hSef-b isoform uses the CUG translation initiation codon in vitro and in HEK 293 cell cultures—To characterize the hSef-b product and elucidate whether the alternative initiation codon in hSef-b can serve as a translation start site, a cDNA fragment encoding the entire ORF of hSef-b (SEQ ID NO:4) was subcloned into the either pcDNA3.1/Hygro or in pcDNA3.1/myc-His(+)B eukaryotic expression vector (Invitrogen) which contains the myc epitope-tag at the carboxyl terminus of the recombinant hSef-b coding sequence allowing the detection of the translation product with the α-myc antibody. As is shown in FIG. 2a, expression of the pcDNA3.1/myc-His-hSef-b DNA construct in HEK 293 cells revealed a single protein product with a molecular weight of about 80 kDa. Similarly, when the hSef-b cDNA was cloned into the pcDNA3.1/Hygro vector (Invitrogen) and was subjected to an in vitro transcription-translation in the presence of [³⁵S]-methionine, a major product with a similar molecular weight (˜80 kDa) was obtained (FIG. 2b). These findings strongly suggest that the CUG codon functions as a major translation start site in the hSef-b isoform.

Human Sef-b is a putative intracellular protein—As is mentioned hereinabove, and depicted in FIG. 1, both hSef isoforms have 8 potential N-linked glycosylation sites. Thus, although hSef-a has a predicted size of 83 kDa, when the coding sequence of hSef-a was expressed in HEK 293 cells, a broad band with an average molecular weight of 120 kDa was observed (FIG. 2a), suggesting massive post-translational glycosylations. On the other hand, the similarity between the apparent (80 kDa, FIG. 2a) and the predicted (i.e., 78 kDa) molecular weight of hSef-b suggests that the hSef-b isoform does not undergo any significant post-translational modifications. To test the hypothesis that hSef-b, unlike hSef-a, is not subjected to post-translational glycosylations, transfected HEK 293 cells were treated with tunicamycin, an inhibitor of N-linked glycosylation, and the molecular weight of the recombinant proteins was examined on an SDS-PAGE. While tunicamycin treatment resulted in a reduced molecular weight of the hSef-a isoform, such a treatment had no effect on the molecular weight of the hSef-b product (data not shown).

Altogether, these results indicate that hSef-a, but not hSef-b, is a glycoprotein synthesized in the classical secretory pathway, and further suggest that the hSef-b product is an intracellular protein.

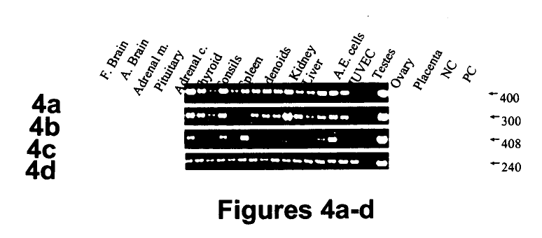

Cellular localization of the human Sef isoforms—To further test the possibility that hSef-b is an intracellular protein, transfected HEK 293 cells expressing each of the human Sef isoforms were subjected to immunofluorescence analysis. Detection was performed using antibodies directed against the myc epitope-tag fused to the hSef products or antibodies directed against the carboxyl-terminus of hSef proteins. As shown in FIGS. 3a-d, while hSef-a was localized to the cell surface (in agreement with prior art findings Tsang, M. et al., 2002) the hSef-b protein was detected in the cytosol of the transfected cell. These findings demonstrate that the hSef-b product is a cytosolic protein.

Altogether, these results demonstrate that the hSef gene undergoes alternative splicing which result in at least two distinct hSef isoforms (hSef-a and b). These results further show that while hSef-a is a heavily glycosylated, membrane protein, the hSef-b isoform apparently lacks any post-translational glycosylation modifications and is localized to the cytosol.

### Example 2

### The Expression of Human Sef Isoforms Is Differentially Regulated

To further understand the role of the newly identified hSef-b isoform, the expression pattern and function of the hSef isoforms were determined, as follows.

Experimental Results

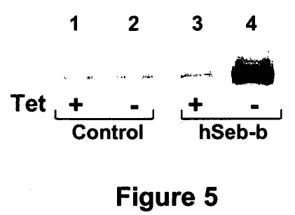

Tissue type specific expression of human Sef isoforms—The pattern of expression of hSef isoforms in a variety of human tissues and cell lines was examined by RT-PCR. Primers were designed to amplify a region common to both hSef transcripts or to specifically amplify each transcript. All 16 samples examined were positive for Sef transcripts when amplified with the primers from the common region of Sef isoforms (FIG. 4a). As is shown in FIG. 4b, hSef-a transcript was differentially expressed in 15 samples; HSef-a transcript was highly expressed in both fetal and adult brain, pituitary, tonsils, spleen, adenoids, fetal kidney, liver, testes and ovary, and moderate levels were detected in primary aortic endothelial cells, human umbilical vein endothelial cells (HUVEC) and adrenal medulla. As is further shown in FIG. 4b, low levels of hSef-a were observed in adrenal cortex, barely detected in placenta and completely absent in thyroid. In contrast, as is shown in FIG. 4c, hSef-b transcript was highly expressed in thyroid and testes; moderately expressed in pituitary, fetal brain and HUVEC cells; remaining tissues were either negative or expressed barely detectable levels of the hSef-b transcript. These findings demonstrate a unique expression pattern of hSef isoforms in a variety of tissues and suggest that the expression of the human Sef isoforms is regulated at the level of splicing or mRNA stability.

### Example 3

### Human Sef-B Isoform Inhibits Proliferation of NIH/3T3 Cells and Interferes with Human HEK 293 Cell Growth

The different biochemical properties and subcellular localization of the two hSef isoforms raised the question whether hSef-b can inhibit FGF biological activity similar to hSef-a. NIH/3T3 cells were chosen to study the effect of hSef-b on biological responses to FGFs since they proliferate in response to various members of the FGF family, and have been extensively utilized as a model to study oncogenesis, regulation of cell proliferation and growth factor-mediated signaling. In addition, preliminary RT-PCR analyses revealed that NIH/3T3 cells express the mouse-Sef gene (data not shown). To study the effect of hSef-b on NIH/3T3 proliferation, cells were transfected with an expression vector containing the hSef-b coding sequence and the effect of hSef-b expression on colony number and proliferation was studied, as follows.

Experinental results

Human Sef-b isoform reduces the number of NIH/3T3 colonies—The general effect of hSef-b on the growth of NIH/3T3 cells was studied as follows. Cells were stably transfected with either the pcDNA/hSef-b or an empty vector (i.e., a pcDNA without the hSef-b insert) and one day following transfection the cells were diluted (1:25) and were further cultured for 2-3 weeks in the presence of hygromycin B (marker selection), following which resistant colonies were counted. As is shown in Table 1, hereinbelow, a significant decrease (75%) in the number of colonies was observed in cells transfected with the hSef-b expression vector as compared with cells transfected with the empty vector.

TABLE 1
Number of colonies in cell stably transfected with hSef-b

| vector | No. of colonies | Ratio (%) |
|---|---|---|
| Empty vector | 110 | 100 |
| hSef-b | 28 | 25.9 |

Table 1: NIH 3T3 cells were stably transfected with either an expression vector (pCDNA3.1) containing the hSef-b coding sequence (SEQ ID NO: 4) or with the pCDNA3.1 vector alone (empty vector) and 2-3 weeks following transfection in the presence of the hygromycin B selection the number of colonies (five plates for each vector) was counted. Results represent the average of three independent experiments.

These results clearly demonstrate that similarly to hSef-a, hSef-b isoform inhibits NIH/3T3 cell growth.

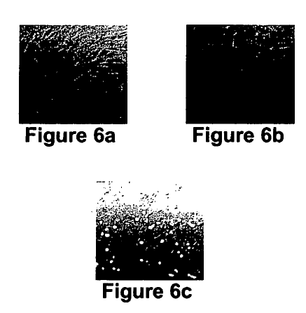

Establishment of tet-off hSef-b NIH 3T3 cells lines—To understand the mechanisms leading to the inhibitory effect of hSef-b on the number of colonies of NIH 3T3 cells and to study the effect of hSef-b on FGF mediated signaling, NIH 3T3 stable cell lines in which the expression of hSef-b is regulated by tetracycline (tet) were established. S2 cells (NIH 3T3) were co-transfected with the pTet splice-hSef-b or an empty vector (i.e., the pTet splice alone, control cells) and the pTK-Hyg vector and were cultured for three weeks in the presence of hygromycin. Clones that did not express detectable levels of hSef-b in the presence of tetracycline were chosen for further analysis. Maximal level of hSef-b protein was obtained 16 hours following removal of tetracycline (FIG. 5, and data not shown).

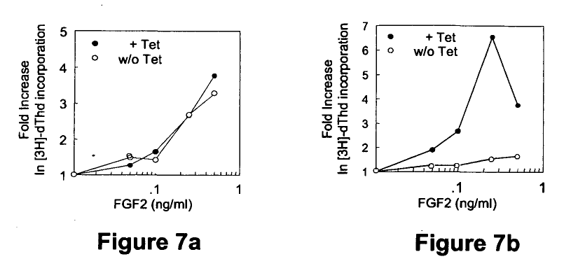

Expression of hSef-b in NIH/3T3 cells does not affect the level of apoptosis—The inhibitory effect of hSef-b on colony formation could have resulted from either induction of apoptosis or inhibition of cell growth. The effect of hSef-b on apoptosis was tested using the TUNEL staining. Stable transfectants of hSef-b NIH 3T3 cells were grown for 48 hours in the presence or absence of tetracycline, or in the absence of tetracycline and serum, following which the cells were washed and fixed and were subjected to a TUNEL assay. As is shown in FIGS. 6a-c, while apoptotic cells were readily detected in NIH 3T3 cells grown in the absence of tetracycline and serum (FIG. 6c), no significant level of apoptosis was observed in NIH 3T3 grown in the presence (i.e., when hSef-b expression is downregulated) or absence (i.e., when hSef-b expression is upregulated) of tetracycline (FIGS. 6a and 6b, respectively).

Thus, these results demonstrate that over-expression of hSef-b in cells doesn't affect apoptotic processes in the cells.

Over-expression of human Sef-b affects the growth of human HEK 293 cells—When HEK 293 cells were transiently transfected with the hSef-b expression vector the morphology of the cells was changed (i.e., more round cells) and the cells came easily off the plate (data not shown). These results suggest that hSef-b affects the normal growth of human cells and likely causes cell death.

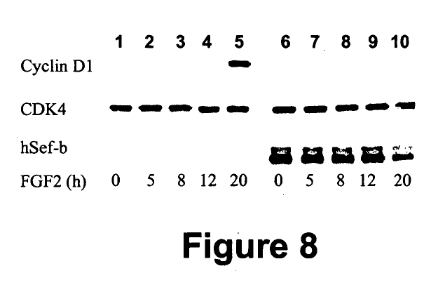

Human Sef-b inhibits NIH 3T3 growth via inhibition of the FGF2 mitogenic activity—To test the possibility that expression of hSef-b involves in FGF2 mitogenic activity, [³H]-thymidine incorporation assays were employed. Confluent cultures of control or hSef-b-expressing cells were serum starved (for 24) and were grown in the presence or absence of tetracycline, following which the level of [³H]-thymidine incorporation was measured in the presence of increasing concentrations of FGF2. As is shown in FIGS. 7a-b, while in control cells [³H]-thymidine incorporation increased along with the increase of FGF2, in hSef-b-expressing cells, at 0.7 ng/ml FGF2, [³H]-thymidine incorporation was reduced. These results demonstrate that over-expression of hSef-b strongly inhibits the mitogenic activity of FGF2.

Altogether, these findings indicate that hSef-b acts by restricting cell division and not by inducing apoptosis in NIH/3T3 cells.

### Example 4

### Human Sef-B Prevents Activation of Erk1/2Map-Kinases

To further understand the mechanisms leading to hSef-b-mediated inhibition of cell growth the present inventor has studies the level of Cyclin-D1 which regulates S-phase entry (31), as well as the level of Erk1/2 MAP-kinases, pkB/Akt and p38 MAP-kinase, which are known to regulate cyclin D1 levels by transcriptional and post-translational mechanisms (31, 32). Erk1/2 MAP-kinases enhance Cyclin D1 expression and regulate the assembly of cyclin-CDK complex, whereas pkB/Akt regulates the turnover of cyclin D1 protein (31, 32). The p38 MAP-kinase can inhibit Cyclin D1 expression in a cell type dependent manner. p38 MAP-kinase has been generally associated with cellular response to stress, but several reports suggest its involvement in cellular responses to growth factors including FGF2 (22, 23).

Experimental Results

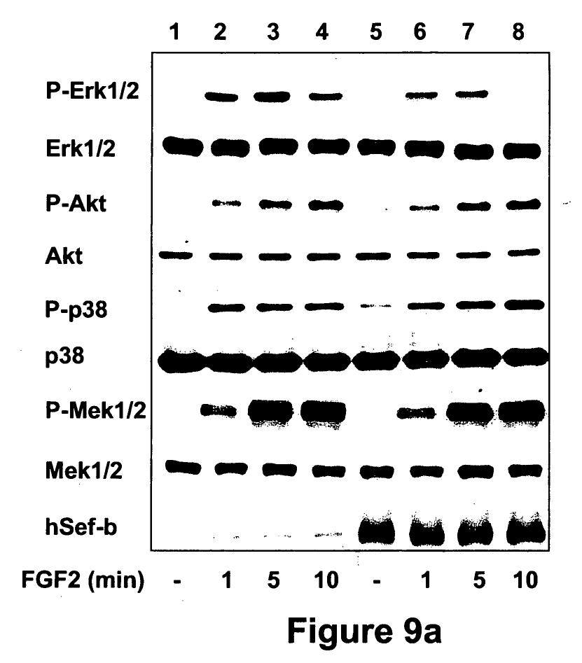

The hSef-b isoform reduces the level of cyclin D1—Sef-inducible cell lines were utilized to explore the mechanism underlying hSef-b inhibition of cell division. Cyclin D1 protein levels were evaluated at different time intervals post FGF2 growth-factor stimulation. As is shown in FIG. 8, while 20 hours following growth factor stimulation the cyclin D1 protein was readily observed in cells cultured in the presence of tetracycline, hSef-b-expressing cells failed to express cyclin D1. Since the levels of cyclin dependent kinase 4 (CDK4) do not fluctuate during the cell cycle (31), the level of CDK4 was examined as a probe for protein levels in each time point. CDK4 levels were similar in all time points in cultures grown in the presence or absence of tetracycline (FIG. 8). In addition, the levels of cyclin D1 remained unchanged in the control cells grown with or without tetracycline (data not shown).

Human Sef-b isoform prevents the activation of Erk1/2 MAP-kinases—Stimulation over time of control cells and hSef-b expressing cells revealed that hSef-b inhibited the activation of Erk1/2 MAP-kinases (phosphorylated Erk1/2) whereas total Erk1/2 levels remained unaltered (Compare P-Erk1/2 (activated) and Erk1/2 in FIGS. 9a and b). On the other hand, HSef-b had no effect on FGF2 induced activation of pkB/Akt or p38 MAP-kinase (FIG. 9a).

Expression of hSef-b does not affect MAP-kinase kinase level and state of activation—To further localize the site of hSef-b action, its effect on the activation of the dual specificity MAP-kinase kinase (MEK1/2), which phosphorylate Erk1/2 was examined in response to growth factor stimulation. As is shown in FIG. 9a, hSef-b had no effect on MEK1/2 activation in FGF2 stimulated cells, suggesting that the MAPK signaling pathway is blocked at the level or downstream of MEK.

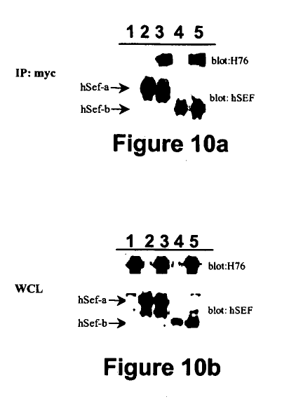

The hSef-b protein associates with FGFR1—Prior studies utilizing co-immunoprecipitation assays demonstrated the association of the cell surface Sef isoform with several members of the FGFR family. Moreover, the site of interaction between the cell surface Sef and FGFR was mapped to the intracellular domain (8, 10, 13). Since as is shown in Example 1 hereinabove, the intracellular domain of hSef-a is shared in common with the hSef-b isoform, the ability of hSef-b to form a complex with FGFR1 was tested. To this end, HEK293 cells were transiently transfected with myc-tagged Sef constructs in the presence or absence of FGFR1. As shown in FIGS. 10a-b, FGFR1 co-immunoprecipitated with hSef-a in the absence of ligand stimulation, in agreement with published data (8, 10, 13). Human Sef-b, notwithstanding its lower expression levels compared to hSef-a, efficiently associated with FGFR1 but not with EGF receptor (FIGS. 10a-b and data not shown). These results lend further support to the importance of the C-terminal domain of Sef for the association with FGFRs.

### Example 5

### HSef-B Inhibits the Mitogenic Activity of FGF1, FGF2, FGF4, and PDGF

The subcellular localization of hSef-b may extend the repertoire of receptor tyrosine kinases (RTKs) that it can inhibit. To explore this hypothesis, the effect of hSef-b on mitogenic activity of additional members of the FGF family, as well as a subset of other RTK ligands, and serum was examined, as follows.

Experimental Results

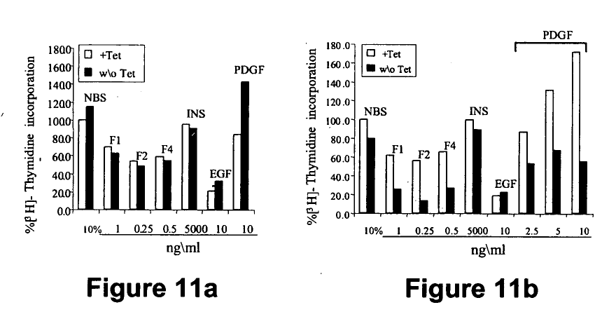

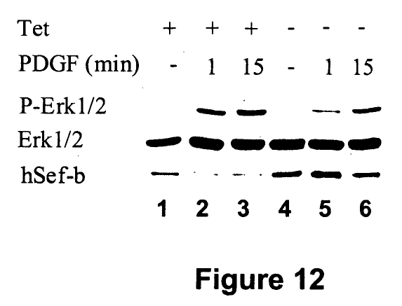

A dose response curve was performed for each ligand, and representative results are shown in FIGS. 11a-b. In addition to FGF2, hSef-b inhibited the mitogenic activity of FGF1 and FGF4 (80% inhibition of FGF2, and 60% inhibition of FGF1 or FGF4) as well as the activity of platelet derived growth factor (PDGF) (40, 57 and 68% inhibition at 2.5, 5 and 10 ng/ml ligand, respectively). These results are in contrast the lack of effect of Sef-a on PDGF signaling (10). Inhibition of PDGF was accompanied with reduction in the activation of Erk1/2 MAPK (FIG. 12). By contrast, hSef-b had little or no effect on the activity of serum, insulin or EGF (FIG. 11b). The mitogenic activity of serum and each of the other growth factors was similar in control cultures grown in the presence or absence of tetracycline (FIG. 11a).

### Example 6

### Identification of Additional Human Sef Splice Forms: HSef-C and HSef-D

Experimental Results

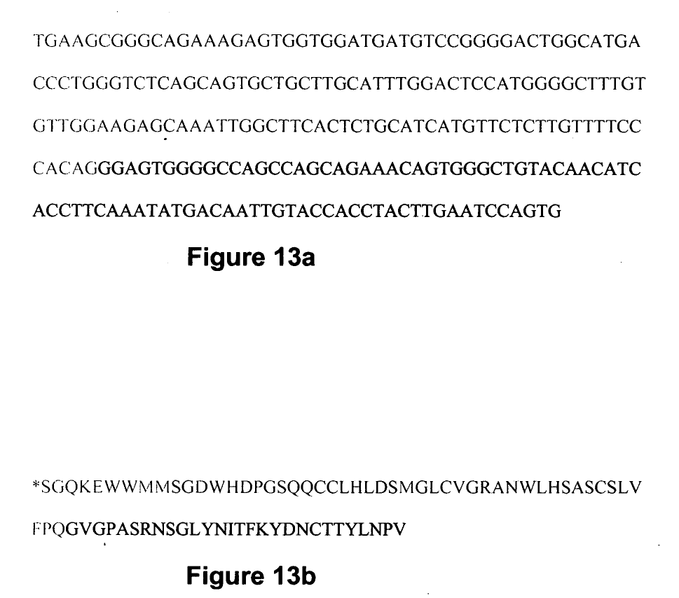

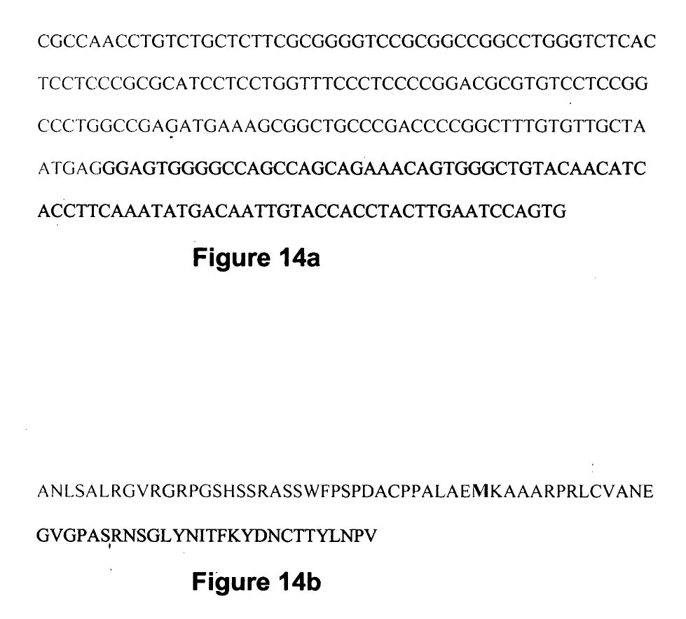

Identification of additional alternative spliced forms of hSef—Using the RACE protocol the present inventor has identified two additional human Sef isoforms from normal human ovary RNA. FIGS. 13a and 14a depict the nucleic acid sequences of hSef-c and hSef-d RACE products, respectively. The predicted amino acid sequences are shown in FIGS. 13b and 14b, for hSef-c and hSef-d, respectively. First strand was using the primer set forth by SEQ ID NO:1, and amplification was performed using the primers set forth by SEQ ID NOs:19 and 3 (for hSef-c) or SEQ ID NOs:21 and 3 (for hSef-d), RT-PCR fragments of approximately 2.2 kb each were identified (data not shown), indicating that the hSef-c and hSef-d are alternative splice forms of the hSef gene. These results suggest the identification of the entire coding region of each isoform. Both isoforms exhibit sequences which are common with the other hSef isoforms (i.e., hSef-a and hSef-b), these include amino acids 50-77 in SEQ ID NO:15 (hSef-c) and amino acids 71-115 in SEQ ID NO:14 (hSef-d). Noteworthy, that while amino acids 21-98 of SEQ ID NO:14 were predicted from the experimentally identified RACE product, the rest of the sequence set forth in SEQ ID NO:14 was predicted using the EST clone (GenBank Accession No. BG149830).

Prediction of open reading frame within the new hSef isoforms—In the coding sequence of human Sef-c isoform there are three potential initiation codons (methionine residues, labeled with a green M in FIG. 13b), and further experiments (e.g., using Edman degradation or Mass Spectrometry) are expected to reveal the active translation initiator. On the other hand, in hSef-d, the predicted initiation codon is the Methionine residue at position 57 of SEQ ID NO:14.

Differential expression of hSef-c in breast and ovary—RT-PCR analysis using primers specific to hSef-c (SEQ ID NOs:19 and 20) revealed differential expression of this isoform; hSef-c is highly expressed in breast and ovary tissues, moderately expressed in brain and to a much lesser extent in fetal kidney (data not shown).

Analysis and Discussion

The results presented in Examples 1-6, hereinabove, indicate that different hSef isoforms are generated via an alternative splicing mechanism. One isoform, hSef-a, is similar to the previously reported Sef from zebrafish and mammals (7-9, 13). This isoform is thought to encode a type I transmembrane protein, and immunofluorescence staining confirmed that hSef-a product is located at the cell surface. On the other hand, in the novel isoform, hSef-b, the leader sequence and the next 8 residues of hSef-a were replaced with 10 different residues. This isoform lacks a signal for secretion and immunofluorescence staining revealed that it is a cytosolic protein. Furthermore, the hSef-b product does not undergo significant post-translational modifications, a property that is typical for proteins that are translated in the cytosolic compartment. Collectively these results demonstrate that alternative splicing differentially influences the subcellular localization of hSef isoforms.

Besides lacking a signal for secretion, the alternate sequence contained a CUG initiation codon instead of the conventional AUG initiation codon. Initiation from this CUG codon is consistent with the apparent molecular weight of both the in vitro and in vivo expressed hSef-b. The utilization of CUG as a translation start site may be responsible for the observed differences in the expression levels of hSef-a and hSef-b products, as non-AUG codons direct less efficient translation initiation (34, 35).

The hSef-b isoform exhibits a restricted pattern of expression compared to hSef-a. It is highly expressed in testes and thyroid and to a much lesser extent in tissues of neuronal origin and primary endothelial cells whereas hSef-a transcript is expressed in all the tissues and primary cells that were examined except for the thyroid. Interestingly, the expression profile of hSef-a parallels that of FGFRs (19, 20, 36-38) suggesting that this isoform regulates a wide array of biological processes where FGFs are implicated. The high levels of hSef-b in thyroid and testes could imply that this isoform regulates unique biological processes and may be more specific to cells of epithelial origin. With respect to FGFs, it may control signaling by specific receptor isoforms. Therefore, an important avenue of future research would be to determine how each Sef isoform affects signaling by the distinct FGFRs.

Human Sef-b inhibited mitogenic response of NIH/3T3 cells to several members of the FGF family, but not to serum, insulin or EGF. Unlike the cell surface Sef (10), hSef-b inhibited PDGF-induced mitogenic response suggesting that intracellular machinery, common to signaling by FGF and PDGF, is affected. Consistent with this is the finding that hSef-b inhibited the Ras/MAPK pathway and reduced cyclin D1 levels. However, hSef-b does not globally affect RTK-induced signaling pathways since it had no effect on FGF induction of the PI 3-kinase or the p38 MAPK pathway, which is consistent with hSef-b action downstream of Ras. Lack of an effect on the PI 3-kinase pathway also correlates with the finding that hSef-b inhibited cell growth but did not lead to an increase in apoptosis. The inhibition of the MAPK pathway and maintenance of PI 3-kinase pathway may allow cells expressing hSef-b to cease proliferation but remain viable upon growth factor stimulation.

Altogether these results restrict hSef-b activity to a narrow window at the level of, or down stream from MEK. Similar to the hSef-a isoform [(8, 10, 13), and present data], the hSef-b isoform can associate with FGFR1 in co-immunoprecipitation assays. Since, unlike hSef-a, the hSef-b isoform is cytosolic, its association with the receptor could function as a mean to bring hSef-b in the vicinity of the components of the Ras/MAPK pathway. Although both isoforms interact with FGFR1, the outcome of this association is not identical because the cell surface Sef isoform inhibits multiple FGF-signaling pathways [(10), and data not shown]. As the entire hSef-b isoform is located inside the cells, its folding must be quite different from that of the hSef-a isoform, and is likely to influence its mode of action. For example, the amino-terminal domain of hSef-b, which also contains the unique hSef-b residues, can interact with proteins in the signaling cascade whereas in hSef-a, this domain is extracellular and is not required for inhibitory activity (8, 10, 13). Alternatively, it could function as an autoregulatory domain that prevents hSef-b from interacting with certain proteins in the signaling cascade. Studies are in progress to address these questions.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### (Additional References Are Cited in the Text)

1. Schlessinger, J. (2000) Cell 103, 211-225.

2. Pawson, T. & Saxton, T. M. (1999) Cell 97, 675-678.

3. Simon, M. A. (2000) Cell 103, 13-15.

4. Hunter, T. (2000) Cell 100, 113-127.

5. Christofori, G. (2003) Nat. Cell Biol. 5, 377-379.

6. Dikic, I. & Giordano, S. (2003) Curr. Opin. Cell Biol. 15, 128-135.

7. Furthauer, M., Lin, W., Ang, S. L., Thisse, B. & Thisse, C. (2002) Nat. Cell Biol. 4, 170-174.

8. Tsang, M., Friesel, R., Kudoh, T. & Dawid, I. B. (2002) Nat. Cell Biol. 4, 165-169.

9. Lin, W., Furthauer, M., Thisse, B., Thisse, C., Jing, N. & Ang, S. L. (2002) Mech. Dev. 113, 163-168.

10. Kovalenko, D., Yang, X., Nadeau, R. J., Harkins, L. K. & Friesel, R. (2003) J. Biol. Chem. 278, 14087-14091.

11. Krasilnikov, M. A. (2000) Biochemistry (Mosc.) 65, 59-67.

12. Scheid, M. P. & Woodgett, J. R. (2001) Nat. Rev. Mol. Cell Biol. 2, 760-768.

13. Yang, R. B., Ng, C. K., Wasserman, S. M., Komuves, L. G., Gerritsen, M. E. & Topper, J. N. (2003) J. Biol. Chem. (on-line)

14. Klint, P. & Claesson-Weish, L. (1999) Front. Biosci. 4:D165-77, D165-77.

15. Powers, C. J., McLeskey, S. W. & Wellstein, A. (2000) Endocr. Relat Cancer 7, 165-197.

16. Ornitz, D. M. & Itoh, N. (2001) Genome Biol. 2, REVIEWS3005.

17. McKeehan, W. L., Wang, F. & Kan, M. (1998) Prog. Nucleic. Acid. Res. Mol. Biol. 59, 135-176.

18. Miki, T., Fleming, T. P., Bottaro, D. P., Rubin, J. S., Ron, D. & Aaronson, S. A. (1991) Science 251, 72-75.

19. Eisemann, A., Ahn, J. A., Graziani, G., Tronick, S. R. & Ron, D. (1991) Oncogene 6, 1195-1202.

20. Ron, D., Reich, R., Chedid, M., Lengel, C., Cohen, O. E., Chan, A. M., Neufeld, G., Miki, T. & Tronick, S. R. (1993) J. Biol. Chem. 268, 5388-5394.

21. Ong, S. H., Hadari, Y. R., Gotoh, N., Guy, G. R., Schlessinger, J. & Lax, I. (2001) Proc. Natl. Acad. Sci. U.S.A 98, 6074-6079.

22. Maher, P. (1999) J. Biol. Chem. 274, 17491-17498.

23. Boilly, B., Vercoutter-Edouart, A. S., Hondermarck, H., Nurcombe, V. & Le, B., X (2000) Cytokine Growth Factor Rev. 11, 295-302.

24. Reich-Slotky, R., Shaoul, E., Berman, B., Graziani, G. & Ron, D. (1995) J. Biol. Chem. 270, 29813-29818.

25. Ron, D., Bottaro, D. P., Finch, P. W., Morris, D., Rubin, J. S. & Aaronson, S. A. (1993) J. Biol. Chem. 268, 2984-2988.

26. Sher, I., Weizman, A., Lubinsky-Mink, S., Lang, T., Adir, N., Schomburg, D. & Ron, D. (1999) J. Biol. Chem. 274, 35016-35022.

27. Ron, D., Tronick, S. R., Aaronson, S. A. & Eva, A. (1 988) EMBO J. 7, 2465-2473.

28. Shockett, P., Difilippantonio, M., Hellman, N. & Schatz, D. G. (1995) Proc. Natl. Acad. Sci. U.S.A 92, 6522-6526.

29. Studier, F. W., Rosenberg, A. H., Dunn, J. J. & Dubendorff, J. W. (1990) Methods Enzymol. 185, 60-89.

30. Shaoul, E., Reich-Slotky, R., Berman, B. & Ron, D. (1995) Oncogene 10, 1553-1561.

31. Sherr, C. J. & Roberts, J. M. (1999) Genes Dev. 13, 1501-1512.

32. Lavoie, J. N., L'Allemain, G., Brunet, A., Muller, R. & Pouyssegur, J. (1996) J. Biol. Chem. 271, 20608-20616.

33. Niehrs, C. & Meinhardt, H. (2002) Nature 417, 35-36.

34. Kozak, M. (1989) Mol. Cell Biol. 9, 5073-5080.

35. Kozak, M. (1991) J. Biol. Chem. 266, 19867-19870.

36. Miki, T., Bottaro, D. P., Fleming, T. P., Smith, C. L., Burgess, W. H., Chan, A. M. & Aaronson, S. A. (1992) Proc. Natl. Acad. Sci. U.S.A. 89, 246-250.

37. Finch, P. W., Cunha, G. R., Rubin, J. S., Wong, J. & Ron, D. (1995) Dev. Dyn. 203, 223-240.

38. Yazaki, N., Hosoi, Y., Kawabata, K., Miyake, A., Minami, M., Satoh, M., Ohta, M., Kawasaki, T. & Itoh, N. (1994) J. Neurosci. Res. 37, 445-452.

1. A method of inhibiting a growth of a solid tumor in a subject, the method comprising administering to the subject an agent capable of upregulating the expression level and/or activity of at least a functional portion of Sef, said at least a functional portion of Sef being capable of inhibiting RTK-mediated cell proliferation, wherein the agent inhibits the growth of the solid tumor in the subject.

2. The method of claim 1, wherein said RTK-mediated cell proliferation is ligand independent.

3. The method of claim 1, wherein said RTK-mediated cell proliferation is ligand-induced.

4. The method of claim 1, wherein said at least a functional portion of Sef is a polypeptide as set forth by SEQ ID NO:6.

5. The method of claim 1, wherein said at least a functional portion of Sef is a polypeptide comprised of amino acids 1-10, 267-707 and/or 288-707 of SEQ ID NO:6.

6. The method of claim 1, wherein said upregulating is effected by at least one approach selected from the group consisting of:
(a) expressing in cells of the subject an exogenous polynucleotide encoding at least a functional portion of Sef; and
(b) increasing expression of endogenous Sef in cells of the subject.

7. The method of claim 6, wherein said exogenous polynucleotide is a nucleic acid construct comprising a polynucleotide at least 90% identical to the polynucleotide sequence set forth in SEQ ID NO:4.

8. The method of claim 6, wherein said exogenous polynucleotide is a nucleic acid construct comprising a polynucleotide as set forth by SEQ ID NO:4.

9. The method of claim 7, wherein said nucleic acid construct further comprises a promoter capable of directing an expression of said polynucleotide in said cells of the subject.

10. The method of claim 9, wherein said promoter is selected from the group consisting of Cytomegalovirus (CMV) promoter, simian virus (SV)-40 early promoter, SV-40 late promoter, metallothionein promoter, murine mammary tumor virus promoter, Rous sarcoma virus (RSV) promoter, and polyhedrin promoter.

11. The method of claim 6, wherein said Sef is a polypeptide at least 90% homologous to a polypeptide set forth by SEQ ID NO:6.

12. The method of claim 6, wherein said Sef is a polypeptide set forth by SEQ ID NO:6.

13. The method of claim 1, wherein said solid tumor is thyroid carcinoma.

14. The method of claim 3, wherein said ligand is selected from the group consisting of FGF, PDGF, VEGF, NGF, insulin, and EGF.

15. The method of claim 1, wherein said RTK-mediated cell proliferation is effected by inhibition of activated Erk1/2 (P-Erk1/2) within said cells.

16. A pharmaceutical composition useful for inhibiting a growth of a solid tumor in a subject comprising, as an active ingredient, an agent capable of upregulating the expression level and/or activity of at least a functional portion of Sef, said at least a functional portion of Sef being capable of inhibiting RTK-mediated cell proliferation, and a pharmaceutically acceptable carrier.

17. The pharmaceutical composition of claim 16, wherein said RTK-mediated cell proliferation is ligand independent.

18. The pharmaceutical composition of claim 16, wherein said RTK-mediated cell proliferation is ligand-induced.

19. The pharmaceutical composition of claim 16, wherein said at least a functional portion of Sef is a polypeptide as set forth by SEQ ID NO:6.

20. The pharmaceutical composition of claim 16, wherein said at least a functional portion of Sef is a polypeptide as set forth by amino acid coordinates 1-10, 267-707 and/or 288-707 of SEQ ID NO:6.

21. The pharmaceutical composition of claim 16, wherein said upregulating is effected by at least one approach selected from the group consisting of:
(a) expressing in cells of the subject an exogenous polynucleotide encoding at least a functional portion of Sef;
(b) increasing expression of endogenous Sef in cells of the subject;
(c) increasing endogenous Sef activity in cells of the subject; and
(d) introducing an exogenous peptide and/or exogenous polypeptide including at least a functional portion of Sef to the subject.

22. The pharmaceutical composition of claim 21, wherein said exogenous polynucleotide is a nucleic acid construct comprising a polynucleotide at least 90% identical to the polynucleotide sequence set forth in SEQ ID NO:4.

23. The pharmaceutical composition of claim 21, wherein said exogenous polynucleotide is a nucleic acid construct comprising a polynucleotide selected from the group consisting of SEQ ID NOs:4, 8, and 9.

24. The pharmaceutical composition of claim 22, wherein said nucleic acid construct further comprises a promoter capable of directing an expression of said polynucleotide in said cells of the subject.

25. The pharmaceutical composition of claim 24, wherein said promoter is selected from the group consisting of Cytomegalovirus (CMV) promoter, simian virus (SV)-40 early promoter, SV-40 late promoter, metallothionein promoter, murine mammary tumor virus promoter, Rous sarcoma virus (RSV) promoter, and polyhedrin promoter.

26. The pharmaceutical composition of claim 21, wherein said Sef is a polypeptide at least 90% homologous (identical+similar) to a polypeptide set forth by SEQ ID NO:6 as determined using the BlastP software where gap open penalty equals 11, gap extension penalty equals 1 and matrix is blosum 62.

27. The pharmaceutical composition of claim 21, wherein said Sef is a polypeptide set forth by SEQ ID NO:6.

28. The pharmaceutical composition of claim 16, wherein said solid tumor is selected from the group consisting of ovarian carcinoma, pancreatic cancer, breast cancer, endometrial carcinoma, brain tumor, adrenal carcinoma, pituitary cancer, thyroid carcinoma, tonsillar carcinoma, spleen cancer, adenoids cancer, kidney cancer, liver cancer, testis cancer, bladder cancer, colon cancer, prostate cancer, bile duct, lung cancer, and stomach cancer.

29. The pharmaceutical composition of claim 18, wherein said ligand is selected from the group consisting of FGF, PDGF, VEGF, NGF, insulin and EGF.

30. The pharmaceutical composition of claim 15, wherein said RTK-mediated cell proliferation is effected by inhibition of activated Erk1/2 (P-Erk1/2) within said cells.
